(19) United States
(12) Patent Application Publication
(10) Pub. No.: US 2006/0252740 A1
(43) Pub. Date: Nov. 9, 2006

(54) Method of treating multiple myeloma using 17-AAG or 17-AG or a prodrug of either in combination with a proteasome inhibitor

(76) Inventors: Robert G. Johnson JR., Lafayette, CA (US); Alison L. Hannah, Sebastopol, CA (US); Gillian F. Cropp, San Francisco, CA (US); Yiqing Zhou, Lafayette, CA (US); J. Michael Sherrill, Danville, CA (US)

Correspondence Address: KOSAN BIOSCIENCES, INC, 3832 BAY CENTER PLACE, HAYWARD, CA, 94588, US

(21) Appl. No.: 11/412,299

(22) Filed: Apr. 26, 2006

Related U.S. Application Data
(60) Provisional application No. 60/676,556, filed on Apr. 29, 2005.
(60) Provisional application No. 60/686,232, filed on May 31, 2005.
(60) Provisional application No. 60/749,190, filed on Dec. 9, 2005.

(51) Int. Cl. A61K 31/395

(52) U.S. Cl. 514/183

(57) ABSTRACT

A method for treating multiple myeloma in a subject by administering to the subject 17-allylamino-17-demethoxy-geldanamycin or 17-amino geldanamycin, or a pro drug of either 17-AAG or 17-AG, in combination with a proteasome inhibitor.

### CROSS-REFERENCE TO RELATED APPLICATIONS

This application claims the benefit under 35 U.S.C. § 119(e) of U.S. Provisional Applications Nos. 60/676,556, filed Apr. 29, 2005; 60/686,232, filed May 31, 2005; and 60/749,190, filed Dec. 9, 2005; the disclosures of which are incorporated herein by reference.

### BACKGROUND OF THE INVENTION

1. Field of the Invention

This invention relates to a method of treating multiple myeloma using 17-allylamino-17-demethoxy-geldanamycin or 17-amino geldanamycin, or a prodrug of either 17-AAG or 17-AG, in combination with a proteasome inhibitor.

2. Description of Related Art

Multiple myeloma (“MM”, also known as myeloma or plasma cell myeloma) is an incurable but treatable cancer of the plasma cell. Plasma cells are an important part of the immune system, producing immunoglobulins (antibodies) that help fight infection and disease. MM is characterized by excessive numbers of abnormal plasma cells in the bone marrow (“BM”) and overproduction of intact monoclonal immuno globulins (IgG, IgA, IgD, or IgE; “M-proteins”) or Bence-Jones protein (free monoclonal light chains). Hypercalcemia, anemia, renal damage, increased susceptibility to bacterial infection, and impaired production of normal immunoglobulin are common clinical manifestations of MM. MM is often also characterized by diffuse osteoporosis, usually in the pelvis, spine, ribs, and skull.

Therapies for MM include chemotherapy, stem cell transplantation, high-dose chemotherapy with stem cell transplantation, and salvage therapy. Chemotherapies include treatment with Thalomid® (thalidomide), bortezomib, Aredia® (pamidronate), steroids, and Zometa® (zoledronic acid). However many chemotherapy drugs are toxic to actively dividing non-cancerous cells, such as of the BM, the lining of the stomach and intestines, and the hair follicles. Therefore, chemotherapy may result in a decrease in blood cell counts, nausea, vomiting, diarrhea, and loss of hair.

Conventional chemotherapy, or standard-dose chemotherapy, is typically the primary or initial treatment for patients with MM. Patients also may receive receive chemotherapy in preparation for high-dose chemotherapy and stem cell transplant. Induction therapy (conventional chemotherapy prior to a stem cell transplant) can be used to reduce the tumor burden prior to transplant. Certain chemotherapy drugs are more suitable for induction therapy than others, because they are less toxic to BM cells and result in a greater yield of stem cells from the BM. Examples of chemotherapy drugs suitable for induction therapy include dexamethasone, thalidomide/dexamethasone, VAD (vincristine, Adriamycin® (doxorubicin), and dexamethasone in combination), and DVd (pegylated liposomal doxorubicin (Doxil®, Caelyx®), vincristine, and reduced schedule dexamethasone in combination).

The standard treatment for MM is melphalan in combination with prednisone (a corticosteroid drug), achieving a response rate of 50%. Unfortunately, melphalan is an alkylating agent and is less suitable for induction therapy. Corticosteroids (especially dexamethasome) are sometimes used alone as MM therapy, especially in older patients and those who cannot tolerate chemotherapy. Dexamethasone is also used in induction therapy, alone or in combination with other agents. VAD is the most commonly used induction therapy, but DVd has recently been shown to be effective in induction therapy. Bortezomib has been approved recently for the treatment of MM, but it is very toxic. However, none of the existing therapies offer a significant potential for a cure.

17-Allylamino-17-demethoxygeldanamycin (“17-AAG”, also sometimes referred to as 17-allylaminogeldanamycin) is a semi-synthetic analog of the naturally occurring compound geldanamycin (Sasaki et al., 1981). Geldanamycin is obtainable by culturing a producing organism, such as Streptomyces hygroscopicus var. geldanus NRRL 3602. Another biologically active geldanamycin derivative is 17-aminogeldanamycin (“17-AG”), which is produced in the human body by metabolism of 17-AAG. 17-AG can also be made from geldanamycin (Sasaki et al. 1979). While geldanamycin and its analogs have been studied intensively as anti-cancer agents in the 1990s (e.g., Sasaki et al., 1981; Schnur, 1995; Schnur et al., 1999), none of them has been approved for anti-cancer use.

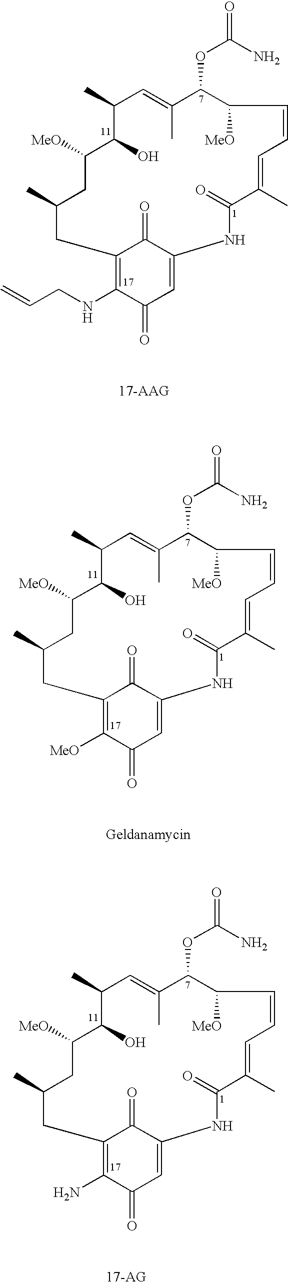

17-AAG and geldanamycin are believed to act by binding to and inhibiting the activity of heat shock protein-90 (“Hsp90”) (Schulte and Neckers, 1998). Hsp90 acts as a chaperone for the normal processing of many cellular proteins (“client proteins”) and is found in all mammalian cells. Stress (hypoxia, heat, etc.) induces a several-fold increase in its expression. There exist other stress-induced proteins (co-chaperones), such as heat shock protein-70 (“Hsp70”), which also play a role in cellular response to and recovery from stress.

In cancer cells, Hsp90 inhibition leads to disruption of the interaction between Hsp90 and its client proteins, such as erbB2, steroid receptors, raf-1, cdk4, and Akt. For example, exposure to 17-AAG results in depletion of erbB2 and destabilization of Raf-1 and mutant p53 in SKBr3 breast cancer cells (Schulte and Neckers, 1998), depletion of steroid receptors in breast cancer cells (Bagatell et al., 2001), depletion of Hsp90 and down-regulation of Raf-1 and erbB2 in MEXF 276L melanoma cells (Burger et al., 2004), depletion of Raf-1, c-Akt, and Erk1/2 in colon adenocarcinoma cells (Hostein et al., 2001), down-regulation of intracellular Bcr-Abl and c-Raf proteins and reduction of Akt kinase activity in leukemia cells (Nimmanapalli et al., 2001), degradation of cdk4, cdk6, and cyclin E in lung cancer cells with wild-type Rb (Jiang and Shapiro, 2002), and depletion of erbB1 (EGFR) and erbB2 (p185) levels in NSCLC cells (Nguyen et al., 2000).

Because of the activity of 17-AAG relative to Hsp90 and other proteins involved in oncogenesis and metastasis of cancer cells, a number of clinical investigators have evaluated its effectiveness as an anti-cancer agent in human clinical trials. From these various trials, the Cancer Therapy Evaluation Program (CTEP) of the National Cancer Institute recommended these Phase 2 dose/schedule regimens for further study: 220 mg/m² (mg per square meter of body surface area of the patient or subject) administered twice weekly for 2 out of 3 weeks, 450 mg/m² administered once a week continuously or with a rest or break, and 300 mg/m² once a week for 3 weeks out of 4 weeks. Results of various clinical trials—almost exclusively with patients having solid tumors—with 17-AAG generally showed limited clinical activity and are summarized below:
(a) A Phase 1 trial in adult patients with solid tumors was conducted in which patients received 17-AAG daily for 5 days every 3 weeks. The starting dose was 10 mg/m² and was escalated to 56 mg/m², with a maximum tolerated dose (“MTD”) and recommended Phase 2 dose defined as 40 mg/m². The protocol was amended to exclude patients with significant pre-existing liver disease, after which patients were treated at doses up to 110 mg/m² on the same schedule. No objective tumor responses were observed. Due to dose limiting reversible hepatotoxicity, the protocol was further amended to dose patients on a twice weekly schedule every other week starting at a dose of 40 mg/m² per day. At daily doses of 40 and 56 mg/m² for 5 days, the peak plasma concentrations were 1,860±660 and 3,170±1,310 nM, respectively. For patients treated at 56 mg/m² average AUC values for 17-AAG and 17-AG were 6,708 and 5,558 nM*h, respectively, and average t_{1/2} 3.8 and 8.6 hours, respectively. Clearances of 17-AAG and 17-AG were 19.9 and 30.8 L/h/m², respectively, and V_{Z} values were 93 and 203 L/m², respectively (Grem et al., 2005).
(b) In a second Phase 1 trial, patients with advanced solid tumors received 17-AAG on a daily×5 schedule at a starting dose of 5 mg/m². At the 80 mg/m², dose limiting toxicities (hepatitis, abdominal pain, nausea, dyspnea) were observed but dose escalations nevertheless were continued until the dose reached 157 mg/m²/day. Further dose schedule modifications were implemented to allow twice weekly dosing. At the 80 mg/m² dose level, the t_{1/2} was 1.5 hours and the plasma Cₘₐₓ was 2,700 nM. Similarly, for 17-AG the t_{1/2} was 1.75 hours and the Cₘₐₓ was 607 nM. Plasma concentrations exceeded those needed to achieve cell kill (10-500 nM) in in vitro and in vivo xenograft models (Munster et al., 2001).
(c) A Phase 1 trial of 17-AAG was conducted in which patients with advanced solid tumors were treated weekly for 3 out of every 4 weeks at a starting dose of 10 mg/m² with a recommended Phase 2 dose of 295 mg/m². Dose escalations reached a dose of 395 mg/m², at which nausea and vomiting secondary to pancreatitis and grade 3 fatigue were observed. The dosing schedule was amended to allow dosing twice weekly for 3 out of every 4 weeks and twice weekly for 2 out of every 3 weeks. A population pharmacokinetic (PK) analysis was performed on data obtained from this trial. The Vd (volume of distribution) for 17-AAG was 24.2 L for the central compartment and 89.6 L for the peripheral compartment. Clearance values were 26.7 L/h and 21.3 L/h for 17-AAG and 17-AG, respectively. Metabolic clearance indicated that 46.4% of 17-AAG was metabolized to 17-AG. No objective tumor responses have been observed in this trial to date. (Chen et al., 2005).
(d) Another Phase 1 trial in patients with solid tumors and lymphomas was conducted using a weekly dosing for 3 weeks out of a 4 week cycle. The starting dose was 15 mg/m². Dose escalation reached 112 mg/m² without significant toxicity and was continued with an objective of reaching a dose range of “biological” activity. The MTD for weekly 17-AAG was reached at 308 mg/m². No objective tumor responses have been observed to date in this trial, and the levels of Hsp90 client proteins measured were unchanged during therapy. No correlation between chaperone or client protein levels and 17-AAG or 17-AG PK was seen. There was also no correlation between the 17-AAG PK and its clinical toxicity (Goetz et al., 2005).
(e) Another Phase 1 trial was conducted using a once weekly administration schedule, including 11 patients with metastatic melanoma. The starting dose was 10 mg/m², and dose limiting toxicity was observed at 450 mg/m²/week (grade ¾ elevation of AST). At higher doses (16-450 mg/m²/week) the 17-AAG formulation employed contained 10-40 mL dimethylsulfoxide (DMSO) in a single infusion, which likely contributed to the gastrointestinal toxicity that was observed in the trial. Among the patients treated at 320-450 mg/m², two showed radiologically documented long term stable disease. No complete or partial responses were recorded. At the highest dose level (450 mg/m²) the plasma 17-AAG concentrations exceeded 10 μM and remained above 120 nM for periods in excess of 24 hours. At the highest dose level of 450 mg/m², the mean volume of distribution was 142.6 L, mean clearance was 32.2 L/h, and the mean peak plasma level was 8,998 μg/L. There was a linear correlation between dose and area under the curve (AUC) for the dose levels studied. Pharmacodynamic (PD) parameters were also measured and induction of the co-chaperone protein Hsp70 was observed in 8 of 9 patients treated at 320-450 mg/m²/week. Depletion of client proteins was also observed in tumor biopsies: CDK4 in 8 out of 9 patients and Raf-1 depletion in 4 out of 6 patients at 24 hours. These data indicated that Hsp90 in tumors is inhibited for between 1 and 5 days. (Banerji et al., 2005).

The in vivo anti-MM activity of 17-AAG has been studied using a model of diffuse GFP positive MM lesions in SCID/NOD mice (Mitsiades et al., 2006). Survival analysis showed that treatment significantly prolonged median overall survival, but non-clinical data are frequently not predictive of clinical activity. As discussed above, this has particularly been the case for 17-AAG in solid tumors, where the promise of pre-clinical data has not been borne out in Phase 1 clinical trials.

Thus, despite intensive efforts to develop 17-AAG as an anti-cancer agent, no regulatory agency has approved it for the treatment of any cancer. There remains a need for methods of dosing and administering 17-AAG and prodrugs of 17-AAG (and its metabolic counterpart 17-AG) so that its potential therapeutic benefits can be realized. The present invention provides such methods that are efficacious in the treatment of MM using 17-AAG.

Recently, preclinical and clinical studies have shown that bortezomib (Velcade®, BZ, PS-341) can overcome resistance of MM cells to conventional or high-dose cytotoxic chemotherapy (Hideshima et al., 2001; Mitsiades et al., 2001; Mitsiades et al., 2003) and improve patient outcome in MM. Bortezomib has recently been approved for treatment of relapsed and refractory MM (Richardson et al., 2003a). Pre-clinical studies have also shown that treatment of MM cells with bortezomib triggers significant Hsp90 up-regulation as a major stress response in MM cells. While bortezomib is capable of improving patient outcome, it is however highly toxic.

The present invention provides combination treatments of 17-AAG or 17-AG or a prodrug of either with bortezomib that are efficacious in the treatment of multiple myeloma.

A list references cited herein is provided at the end of this specification. All documents cited herein are incorporated herein by reference as if each such publication or document were specifically and individually incorporated herein by reference.

### BRIEF SUMMARY OF THE INVENTION

The present invention provides methods for treating multiple myeloma (MM) in a subject in need of such treatment, said methods comprising the step of administering to said subject a therapeutically effective dose of 17-AAG or 17-AG or a prodrug of either 17-AAG or 17-AG and a therapeutically effective dose of a proteasome inhibitor, and optionally repeating said step until no further therapeutic benefit is obtained.

In one embodiment, the method comprises the administration of multiple doses of 17-AAG or a prodrug thereof to a subject with MM over a time period of at least 2 weeks, wherein each such dose is in the range of about 100 mg/m² to about 340 mg/m² of 17-AAG or an equivalent amount of a 17-AAG or 17-AG prodrug. In one embodiment, the dose is about 340 mg/m² of 17-AAG or an equivalent amount of a 17-AAG or 17-AG prodrug. In one embodiment, this dose is administered twice weekly for at least two weeks. In one embodiment, this dose is administered twice weekly for at least two weeks in a three week period, which rate of dosing per three week period is called a cycle, and multiple cycles of such treatment are administered to the MM patient.

In one embodiment, the therapeutically effective dose of 17-AAG or a prodrug of 17-AAG is a dose that results in an AUCₜₒₜₐₗ of 17-AAG per dose in the range of about 2,300 to 19,000 ng/mL*h. In one embodiment, this dose is administered at a rate and frequency such that the Cₘₐₓ of 17-AAG (or the prodrug) does not exceed 9,600 ng/mL (or the molar equivalent of the prodrug). In one embodiment, this dose is administered at a rate and frequency such that the Cₘₐₓ of 17-AAG is greater than 1,300 ng/mL. In one embodiment, this dose is administered at a rate and frequency such that the Cₘₐₓ of 17-AAG is greater than 1,800 ng/mL. In one embodiment, this dose is administered at a rate and frequency such that the Cₘₐₓ of 17-AAG is greater than 1,300 but does not exceed 9,600 ng/mL. In one embodiment, this dose is administered at a rate and frequency such that the Cₘₐₓ of 17-AAG is greater than 1,800 but does not exceed 9,600 ng/mL.

In one embodiment, the therapeutically effective dose of 17-AG or a prodrug of 17-AG (which prodrug includes 17-AAG) is a dose that results in an AUCₜₒₜₐₗ of 17-AG per dose in the range of about 800 to about 17,000 ng/mL*h. In one embodiment, this dose is administered at a rate and frequency such that the Cₘₐₓ of 17-AG does not exceed 1,400 ng/mL. In one embodiment, this dose is administered at a rate and frequency such that the Cₘₐₓ of 17-AG is greater than 140 ng/mL. In one embodiment, this dose is administered at a rate and frequency such that the Cₘₐₓ of 17-AG is greater than 230 ng/mL. In one embodiment, this dose is administered at a rate and frequency such that the Cₘₐₓ of 17-AG is greater than 140 but does not exceed 1,400 ng/mL. In one embodiment, this dose is administered at a rate and frequency such that the Cₘₐₓ of 17-AG is greater than 230 but does not exceed 1,400 ng/mL.

In one embodiment, the therapeutically effective dose of 17-AAG, a prodrug of 17-AAG, 17-AG, or a prodrug of 17-AG is a dose that results in a combined AUCₜₒₜₐₗ of 17-AAG and 17-AG per dose in the range of about 3,500 to 35,000 ng/mL*h. In one embodiment, this dose is administered at rate and frequency such that the Cₘₐₓ of 17-AAG does not exceed 9,600 ng/mL and/or the Cₘₐₓ of 17-AG does not exceed 1,400 ng/mL. In one embodiment, this dose is administered at a rate and frequency such that the Cₘₐₓ of 17-AAG is greater than 1,300 ng/mL and/or the Cₘₐₓ of 17-AG is greater than 140 ng/mL. In one embodiment, this dose is administered at a rate and frequency such that the Cₘₐₓ of 17-AAG is greater than 1,800 ng/mL and/or the Cₘₐₓ of 17-AG is greater than 230 ng/mL. In one embodiment, this dose is administered at a rate and frequency such that the Cₘₐₓ of 17-AAG is greater than 1,300 but does not exceed 9,600 ng/mL and/or the Cₘₐₓ of 17-AG is greater than 140 but does not exceed 1,400 ng/mL. In one embodiment, this dose is administered at a rate and frequency such that the Cₘₐₓ of 17-AAG is greater than 1,800 but does not exceed 9,600 ng/mL and/or the Cₘₐₓ of 17-AG is greater than 230 but does not exceed 1,400 ng/mL.

In one embodiment, the therapeutically effective dose of 17-AAG or a prodrug of 17-AAG is a dose that results in a Terminal ty, of 17-AAG in the range of 1.6 to 5.6 h. In one embodiment, the therapeutically effective dose of 17-AAG or a prodrug of 17-AAG is a dose that results in a Terminal t_{1/2} of 17-AAG in the foregoing range and an AUCₜₒₜₐₗ of 17-AAG per dose in the range of about 2,300 to about 19,000 ng/mL*h.

In one embodiment, the therapeutically effective dose of 17-AG or a prodrug of 17-AG is a dose that results in a Terminal ty, of 17-AG in the range of 3.7 to 9.1 h. In one embodiment, the therapeutically effective dose of 17-AG or a prodrug of 17-AG is a dose that results in a Terminal t_{1/2} of 17-AG in the foregoing range and an AUCₜₒₜₐₗ of 17-AG per dose in the range of about 800 to about 17,000 ng/mL*h.

In one embodiment, the therapeutically effective dose of 17-AAG or a prodrug of 17-AAG is a dose that results in a Volume of distribution V_{Z} of 17-AAG in the range of 56 to 250 L. In one embodiment, the therapeutically effective dose of 17-AAG or a prodrug of 17-AAG is a dose that results in a Volume of distribution V_{Z} of 17-AAG in the foregoing range and an AUCₜₒₜₐₗ of 17-AAG per dose in the range of about 2,300 to 19,000 ng/mL*h.

In one embodiment, the therapeutically effective dose of 17-AAG or a prodrug of 17-AAG is a dose that results in a Clearance in the range of 13 to 85 L/h. In one embodiment, the therapeutically effective dose of 17-AAG or a prodrug of 17-AAG is a dose that results in a Clearance of 17-AAG in the foregoing range and an AUCₜₒₜₐₗ of 17-AAG per dose in the range of about 2,300 to about 19,000 ng/mL*h.

In one embodiment, the therapeutically effective dose of 17-AAG or a prodrug of 17-AAG is a dose that results in a V_{SS} in the range of 96 to 250 L. In one embodiment, the therapeutically effective dose of 17-AAG or a prodrug of 17-AAG is a dose that results in a V_{SS} of 17-AAG in the foregoing range and an AUCₜₒₜₐₗ of 17-AAG per dose in the range of about 2,300 to about 19,000 ng/mL*h.

In one embodiment, the 17-AAG, 17-AG, or a prodrug of either 17-AAG or 17-AG, and the proteasome inhibitor are each administered in separate pharmaceutical formulations. In another embodiment, the 17-AAG, 17-AG, or prodrug of either 17-AAG or 17-AG, and proteasome inhibitor are in the same pharmaceutical formulation. The pharmaceutical formulations each optionally further comprise a pharmaceutically acceptable carrier or diluent.

In one embodiment, the proteasome inhibitor is bortezomib. In one embodiment, each dose of 17-AAG, 17-AG, or prodrug of either 17-AAG or 17-AG, is administered over 90 or 120 minutes as an infusion, and each dose of the bortezomib is administered as an intravenous rapid bolus of 3 to 5 seconds. In one embodiment, each dose of the bortezomib is administered prior to each dose of 17-AAG, 17-AG, or a prodrug of either 17-AAG or 17-AG. In one embodiment, the method comprises the administration of multiple doses of bortezomib to a patient with MM over a time period of at least 2 weeks, wherein each such dose is at least 1 mg/m² or in the range of about 1 mg/m² to about 1.3 mg/m² of bortezomib.

In one embodiment, the method comprises the administration of multiple doses of bortezomib and 17-AAG, 17-AG, or prodrug of either 17-AAG or 17-AG to a subject with MM over a time period of at least 2 weeks, wherein each such dose of bortezomib is at least 1 mg/m² or in the range of about 1 to about 1.3 mg/m² of bortezomib, and each dose of 17-AAG is at least 100 mg/m² of 17-AAG (or an equivalent amount of 17-AG or prodrug of either 17-AAG or 17-AG) or in the range of about 100 to about 340 mg/m² of 17-AAG (or an equivalent amount of 17-AG or prodrug of either 17-AAG or 17-AG). In a preferred embodiment, the method comprises administering multiple doses of bortezomib and 17-AAG, 17-AG, or prodrug of either 17-AAG or 17-AG to a subject with MM over at least 2 weeks, wherein each such dose of bortezomib is at least 1 mg/m² or in the range of about 1 to about 1.3 mg/m², and each dose of 17-AAG, 17-AG, or prodrug of either 17-AAG or 17-AG is at least 150 mg/m² of 17-AAG (or an equivalent amount of 17-AG or prodrug of either 17-AAG or 17-AG) or in the range of about 150 to about 340 mg/m² of 17-AAG (or an equivalent amount of 17-AG or prodrug of either 17-AAG or 17-AG).

### BRIEF DESCRIPTION OF THE DRAWING(S)

FIG. 1 shows the plasma concentration of 17-AAG and 17-AG versus time for dose level 1 (0.7 mg/m² bortezomib and 100 mg/m² 17-AAG), with mean and standard deviation (SD) for Day 1 and Day 11 combined.

FIG. 2 shows the plasma concentration of 17-AAG and 17-AG versus time for dose level 2 (1.0 mg/m² bortezomib and 100 mg/m² 17-AAG), with mean and SD for Day 1 and Day 11 combined.

FIG. 3 shows the plasma concentration of 17-AAG and 17-AG versus time for dose level 3 (1.0 mg/m² bortezomib and 150 mg/m² 17-AAG), with mean and SD for Day 1 and Day 11 combined.

FIG. 4 shows the plasma concentration of 17-AAG and 17-AG versus time for dose level 4 (1.3 mg/m² bortezomib and 150 mg/m² 17-AAG), with mean and SD for Day 1 and Day 11 combined.

FIG. 5 shows the AUCₜₒₜₐₗ of 17-AAG and 17-AG for individual patients.

FIG. 6 shows the total exposure (the sum of AUCₜₒₜₐₗ (17-AAG) and AUCₜₒₜₐₗ (17-AG)) for individual patients.

FIG. 7 shows the percent reduction of serum M-spike, total IgA, and urine M-protein in a patient (Patient 201).

FIG. 8 shows the percent reduction of serum M-spike and total IgG in a patient (Patient 204).

FIG. 9 shows the percent reduction of serum M-spike in a patient (Patient 307).

FIG. 10 shows the percent reduction of serum M-spike and urine M-protein in a patient (Patient 308).

FIG. 11 shows the percent reduction of 20S proteasome activity following doses of 0.7 mg/m² bortezomib and 100 mg/m² 17-AAG; 1.0 mg/m² bortezomib and 100 mg/m² 17-AAG; 1.0 mg/m² bortezomib and 150 mg/m² 17-AAG; and 1.3 mg/m² bortezomib and 150 mg/m² 17-AAG (Treatment Cycle 1, Day 11).

FIGS. 12A and 12B show the induction of apoptosis and reduction in AKT levels in CD138⁺ myeloma cells after four infusions of 17-AAG.

### DETAILED DESCRIPTION OF THE INVENTION

Definitions

To aid in understanding and practice of the present invention, definitions for certain terms used herein are provided below.

In describing the invention, a concentration of 17-AAG is defined to include a molar equivalent concentration of a prodrug of 17-AAG.

In describing the invention, a concentration of 17-AG is defined to include a molar equivalent concentration of a prodrug of 17-AG.

“Adverse effects” are as defined in National Cancer Institute (2003).

A “dose limiting toxicity” (DLT) is defined as any of the following clinical toxicities, referencing National Cancer Institute (2003). Hematologic toxicities comprise: (1) Grade 4 neutropenia (absolute neutrophil count (ANC)<0.5×10⁹/L) for more than 5 consecutive days, or febrile neutropenia (ANC<1.0×10⁹/L, fever>38.5° C.), (2) Grade 4 thrombocytopenia (platelets<25.0×10⁹/L or bleeding episode requiring platelets transfusion), and/or Grade 4 anemia (Hemoglobin<6.5 g/dl). Non-Hematologic toxicities comprise: (1) any ≧Grade 3 non-hematologic toxicity (except Grade 3 injection site reaction, alopecia, anorexia, fatigue), (2) nausea, diarrhea and/or vomiting of Grade≧3 despite the use of maximal medical intervention and/or prophylaxis, and/or (3) treatment delay of more than 4 weeks due to prolonged recovery from a drug-related toxicity.

“Complete response (CR)” is defined on the basis of negative immunofixation (“IF”) on both serum and urine, maintained for at least 6 weeks. A bone marrow aspirate (“BMA”) containing <5% plasma cells can be used to confirm a CR. A trephine biopsy is performed, and the results indicate <5% plasma cells. In non-secretory myeloma, the marrow biopsy is repeated after a 6-week interval to confirm a CR. No increase in the size or number of lytic lesions should occur (development of a compression fracture does not exclude response), with disappearance of soft tissue plasmacytomas.

“KPS performance status” is as defined in Table 1, which also provides a comparison against the ECOG Scale.

TABLE 1
KPS Performance Status

| Karnofsky Scale | | ECOG Scale | |
|---|---|---|---|
| Normal, no complaints | 100 | Fully active, able to carry on all pre- | 0 |
| | | disease performance without | |
| | | restriction | |
| Able to carry on normal | 90 | | |
| activity, minor signs or symptons of disease | | | |
| Normal activity with effort | 80 | Restricted in physically strenuous | 1 |
| | | activity but ambulatory and able to | |
| | | carry out work of a light or | |
| | | sedentary nature (e.g., office work | |
| | | or light house work) | |
| Unable to carry on normal activity or perform | 70 | | |
| active work; cares for | | | |
| self | | | |
| Requires occasional assistance but | 60 | Ambulatory and capable of all self- | 2 |
| is able to care for most own needs | | care but unable to carry out any | |
| | | work activities; up and about more | |
| | | than 50% of waking hours | |
| Requires considerable assistance | 50 | | |
| and frequent medical care | | | |
| Disabled; requires special medical | 40 | Capable of only limited self-care, | 3 |
| care and assistance | | confined to bed or chair more than | |
| | | 50% of waking hours | |
| Severely disabled; hospitalization | 30 | | |
| indicated although death not | | | |
| imminent | | | |
| Very sick; hospitalized and active | 20 | Completely disabled; cannot | 4 |
| | | perform any self-care; totally | |
| | | confined to bed or chair | |
| Moribund; fatal processes | 10 | | |
| progressing rapidly | | | |
| Dead | 0 | | |

“Minimal response” is defined as one or more of the following: between 25-49% reduction in serum M-protein, maintained for at least six weeks; between 50-89% reduction in urinary light chain excretion which still exceeds 200 mg/24 hours, maintained for at least 6 weeks; for patients with non-secretory myeloma only, between 25-49% reduction in plasma cells in a BMA or a bone trephine biopsy, if biopsy is performed, maintained for at least 6 weeks; between 25-49% reduction in the size of soft tissue plasmacytomas (by radiography or clinical examination); and no increase in the size or number of lytic lesions (development of a compression fracture does not exclude response). (Bladé et al., 1998.)

“No Change” is defined as not meeting the criteria of either minimal response or progressive disease. (Bladé et al., 1998.)

“Partial response (PR)” is defined as occurring in patients in whom some, but not all, of the criteria for CR have been met, including those in whom routine electrophoresis is negative but on whom IF has not been performed. See Bladé et al. (1998) for examples.

“Plateau phase” is defined on the basis of stable paraprotein levels for a minimum of 3 months. Plateau will require observations to be within 25% of the value when response is assessed, a rise above 25% being one of the criteria for disease progression. (Bladé et al., 1998.)

“Progression of disease,” for patients not in CR, is defined as a definite increase in disease activity in patients in partial remission or plateau phase, whereas the term relapse applies to a recurrence of evident disease in patients previously in CR. See Blade et al. (1998) for examples.

“Refractory cancer” means a cancer that has not responded to one or more previous treatment.

“Relapse” means the return of signs and symptoms of cancer after a period of improvement from one or more previous treatment. “Relapse from CR” is defined as one or more of the following: a reappearance of serum or urinary paraprotein on IF or routine electrophoresis, confirmed by at least one further investigation and excluding oligoclonal reconstitution; a greater than 5% plasma cells in a BMA or on trephine bone biopsy; development of new lytic bone lesions or soft tissue plasmacytomas or definite increase in the size of residual bone lesions (development of a compression fracture does not exclude continued response and may not indicate progression); and development of hypercalcemia (corrected serum calcium greater than 11.5 mg/dL) not attributable to any other cause.

“Therapeutically effective dose” means, otherwise indicated, the amount of drug that is required to be administered to achieve the desired therapeutic result.

### Embodiments

The present invention provides important new methods for using 17-AAG or 17-AG and prodrugs that exert their anti-cancer effect through the in vivo formation of 17-AAG or 17-AG to treat MM. The present invention arose in part from the discovery of new methods for dosing and administering 17-AAG to achieve and maintain therapeutically effective blood levels of 17-AAG or its major metabolite 17-AG (or blood levels of 17-AAG added together with 17-AG, as these moieties are equipotent in cellular assays), expressed as AUCₜₒₜₐₗ, Cₘₐₓ, Terminal t_{1/2}, Clearance, Volume of distribution, and/or V_{SS}, without reaching blood levels likely to cause unmanageable toxicity.

In one embodiment, the method of the present invention comprises administering multiple doses of 17-AAG, or a prodrug of 17-AAG, and multiple doses of the proteasome inhibitor, over a period of three weeks. Collectively, these doses over the three week period are called a cycle. A patient may be treated with multiple cycles of therapy. Different cycles, including cycles of longer or shorter duration or involving greater or fewer doses than described specifically herein, can be used to practice the present invention, so long as the therapeutically effective doses described herein are achieved. In one embodiment, four doses are administered per cycle, and a period of 3 to 4 days between each dose. In another embodiment, four doses are administered per cycle, with two doses per week administered for the first two weeks of the three week cycle.

In one embodiment, the therapeutically effective dose is achieved by the administration of multiple doses of 17-AAG, or a prodrug of 17-AAG or 17-AG, in combination with (including separate administration within at least one week of one another) a proteasome inhibitor, to a patient with MM over a time period of at least 3 weeks, wherein such multiple doses result in an AUCₜₒₜₐₗ for 17-AAG per dose of at least 2,300 but does not exceed 19,000 ng/mL*h. In one embodiment, four doses are administered per cycle, with each dose being at least 100 or 150 mg/m², and a period of 3 to 4 days between each dose. In another embodiment, four doses are administered per cycle, with two doses per week administered for the first two weeks of the three week cycle.

Compounds other than 17-AAG or 17-AG can be administered that are converted in vivo to 17-AAG or 17-AG (prodrugs). One type of prodrug is that in which the benzoquinone ring is reduced to a hydroquinone ring, but is metabolized back to a benzoquinone ring in the subject. A specific example of a 17-AAG prodrug is 17-allylamino-18,21-dihydro-17-demethoxygeldanamycin. (Adams et al., 2005). The methods of the present invention therefore include, in one embodiment, a method for treating MM in a patient in need of said treatment, wherein the method comprises the administration of multiple doses of 17-AAG or 17-AG, or a prodrug of 17-AAG or 17-AG, to a subject with MM, over a time period of at least 3 weeks, wherein such multiple doses result in an AUCₜₒₜₐₗ for 17-AG per dose of at least 5,000 but does not exceed 18,000 ng/mL*h. In one embodiment, four doses are administered per cycle, with each dose being at least 150 mg/m², and a period of 3 to 4 days between each dose. In another embodiment, four doses are administered per cycle, with two doses per week administered for the first two weeks of the three week cycle.

Thus, the present invention includes within its scope the use of prodrugs of 17-AAG and the term “administering” encompasses the treatment of MM with a pharmaceutically equivalent amount of compound that converts to 17-AAG or 17-AG in vivo after administration to the subject in need thereof. Conventional procedures for the selection and preparation of suitable prodrug derivatives are described in Wermuth, 2003.

A proteasome inhibitor is any compound that inhibits protein degradation by a proteasome that in combination with a 17-AAG, 17-AG or any prodrug of either 17-AAG or 17-AG is efficacious in treating a subject suffering from MM or that exerts its therapeutic action by a mechanism substantially similar to that of bortezomib. In one embodiment, the proteasome inhibitor is an antineoplastic agent and is a reversible inhibitor of the chymotrypsin-like activity of the 26S proteasome in mammalian cells. The proteasome inhibitor can be natural or synthetic. Suitable natural proteasome inhibitors include, but are not limited to, lactacystin, epoxyketones and TMC-95 cyclic peptides. Example of epoxyketones include, but are not limited to, epoxomicin and eponemycin. Suitable synthetic proteasome inhibitors include, but are not limited to, peptide aldehydes and peptide vinyl sulfones. Example of peptide aldehydes include, but are not limited to, Z-Leu-Leu-Leu-al (MG132), Z-Ile-Glu(Obut)-Ala-Leu-al (PSI), and Ac-Leu-Leu-Nle-al (ALLN). See, e.g., Kisselev and Goldberg (2001) and Richardson et al. (2003b). Examples of proteasome inhibitors include, but are not limited to, PS-519 (Shah et al. (2002)), NPI-0052 (Cusack et al. (2005)), ZL₃VS (Kadlcikova et al. (2004)), AdaAhx3L3VS (Kadlcikova et al. (2004)), efrapeptin (Abrahams, et al. (1996)). In one embodiment, the peptide aldehyde has the aldehyde group replaced with boronic acid to form a peptide boronate. In one embodiment, the peptide boronate is a dipeptide boronic acid. In one embodiment, the dipeptide boronic acid is bortezomib.

Bortezomibis an antineoplastic modified dipeptidyl boronic acid that is a reversible inhibitor of the chymotrypsin-like activity of the 26S proteasome in mammalian cells. The making and using of bortezomib and suitable pharmaceutical formulations and means of administration thereof, are taught in Adams et al. (1998, 2000, 2001, 2003, and 2004) and Gupta (2004). Bortezomib is commercially available under the brand name Velcade® (Millennium Pharmaceuticals, Inc., Cambridge, Mass.) and is approved for the treatment of MM patients who have received at least one prior therapy and have demonstrated disease progression after the preceding therapy. A pharmaceutical formulation comprising bortezomib can comprise about 0.9% saline and 1.0 mg/mL mannitol. A single dosage of bortezomib can be from at least about 0.7 to about 1.3 mg/m². The bortezomib can be administered by injection, with the entire dose is injected within 3 to 5 seconds into the subject by direct injection or intravenous infusion.

The subject in need of treatment, for purposes of the present invention, is typically a human patient suffering from MM, although the methods of the invention can be practiced for veterinary purposes, with suitable adjustment of the unit dose to achieve the equivalent AUCₜₒₜₐₗ or other PK and PD parameters described herein for the particular mammal of interest (including cats, cattle, dogs, horses, and the like). Those of skill in the art of pharmaceutical science know or can readily determine the applicable conversion factors for the species of interest from the present disclosure of the doses and PK parameters for human therapy. Typically, however, the methods will be practiced to benefit human subjects, and those subjects will typically have exhibited some histological evidence of MM, including one or more of the following: M spike in serum or urine, BM plasmacytosis of >30%, anemia, renal failure, hypercalcemia, and/or lytic bone lesions.

In one embodiment, the subject has been diagnosed with Stage III MM under the Durie-Salmon system and exhibits one or more of these symptoms: hemoglobin value<8.5 g/dL, serum calcium value>12 mg/dL, advanced lytic bone lesions (scale 3), high M-component production rate (IgG value>7 g/dL; IgA value>5 g/dl; Bence Jones protein>12 g/24 hour). Alternatively, the has been diagnosed with Stage III MM based on the International Staging System (ISS) system, with serum levels of β-2 microglobulin>5.5 g/dL.

In another embodiment, the subject been diagnosed with Stage II MM under the Durie-Salmon system but does not have Stage III MM and has some but not all of these symptoms: hemoglobin value>10 g/dL, serum calcium value≦12 mg/dL, bone x-ray, normal bone structure (scale 0) or solitary bone plasmacytoma only, low M-component production rate (IgG value<5 g/dL; IgA value<5 g/dL). Alternatively, the subject has been diagnosed under the ISS system with Stage II MM but not Stage III MM and does not have serum levels of β-2 microglobulin<3.5 g/dL and albumin≧3.5 g/dL.

In another embodiment, the patient will have one or more of the following signs or symptoms of MM: an elevated level of serum M protein (such as >3 g/dL), and/or more than 10% of the cells in a BM sample from the subject are plasma cells. In another embodiment, prior to treatment the Karnofsky performance status (KPS) of the patient is at least 70%. In another aspect, the KPS of the patient is at least 60%, 50%, 40%, 30%, 20%, or 10%. In one aspect, the ECOG of the patient is at least 0, 1, 2, or 3.

A therapeutically effective dose of 17-AAG, 17-AG, or a prodrug of either 17-AAG or 17-AG, and a therapeutically effective dose of the proteasome inhibitor are the amounts of 17-AAG, 17-AG, or a prodrug of either 17-AAG or 17-AG, and the proteasome inhibitor, respectively, that is administered in combination at each administration over one treatment cycle to the subject that brings about a therapeutic result. The therapeutic result can be that the rate of the progression or spread of the cancer is slowed or stopped for some period of time. In some patients, the therapeutic result can be partial or complete elimination of MM. In some patients, a therapeutic result will be achieved with one treatment cycle. In other patients, a therapeutic result will be achieved only after multiple cycles of treatments. As those of skill in the art will appreciate, however, there can be no assurance that every MM patient will achieve a therapeutic result with any anti-cancer therapy.

As noted above, in one embodiment, each treatment cycle is three weeks. In other embodiments, other treatment cycle times can be employed, such as two or four weeks (or one month), so long as the equivalent AUCₜₒₜₐₗ or other PK and PD parameters described herein are achieved. The unit dose employed in each cycle is administered at least once and up to eight times per treatment cycle. Typically, the dose is administered two to four times per treatment cycle. In one embodiment, the dose is administered twice weekly for 2 weeks out of each treatment cycle of three weeks. For example, if one starts a cycle at the administration of the first dose, then in one embodiment, the unit dose is administered once or twice in the first two weeks of the treatment cycle and not during the third week. In one embodiment, the dose is administered on days 1, 4, 8, and 11 of each treatment cycle, with day 1 being the day the first dose is administered.

Each unit dose of 17-AAG is a dose of not more than the maximally tolerable dose (“MTD”), which can be defined as the maximum dose at which one or fewer of six subjects undergoing the method of treatment experience hematologic or non-hematologic toxicity not amenable to supportive care. Preferably, the amount of 17-AAG administered is equal to or less than the MTD. Preferably, the amount of 17-AAG administered is one that does not result in unacceptable and/or unmanageable hematologic or non-hematologic toxicity.

The therapeutically effective amount of a unit dose 17-AAG or 17-AG or a prodrug of either is the amount that, after one or more cycles of administration in accordance with this invention, results in a complete response (CR), a partial response (PR), a minimal response (MR), a stable disease (StD) condition, a reduction of serum monoclonal protein (serum M protein), or a reduction of plasma cells in the BM of the subject (Blade et al., 1998), for at least a period of time, such as 3 weeks, 6 weeks, 2 months, 6 months, one year, or several years. In one embodiment, the administration of 17-AAG results in a decrease in serum and/or urine M protein, BM plasmocytosis, alleviation of anemia, alleviation of renal failure, alleviation of hypercalcemia, and/or reduction/alleviation of lytic bone lesions in the MM patient. In one embodiment, some patients will not relapse from a CR or will experience a significant delay in the progression of the disease.

The amount of 17-AAG administered in a single unit dose can range from 100 to 340 mg/m² per dose. Where the 17-AAG is administered twice weekly for two out of every three weeks, the amount of 17-AAG administered ranges from 100 to 340 mg/m² per dose. Preferably, the amount of 17-AAG administered ranges from 150 to 340 mg/m² per dose. The amount of 17-AAG administered may also range from 220 to 340 mg/m² per dose. Those of skill in the art will recognize that the unit dose amounts of 17-AAG or 17-AG prodrugs or 17-AG itself can be calculated from the doses provided herein for 17-AAG and the PK parameters provided for 17-AAG and 17-AG and the molecular weight and relative bioavailability of the prodrug or 17-AG.

The method of the invention can also be described in terms of the amount of 17-AAG administered per treatment cycle. The per cycle amount will typically be greater than 400 mg/m², and more usually will be greater 600 mg/m². Typically the per cycle amount will be at least 880 mg/m². In various embodiments, the amount of 17-AAG administered is at least 600 to 1,360 mg/m² per treatment cycle; 880 to 1,360 mg/m² per treatment cycle; and 1,100 to 1,360 mg/m² per treatment cycle.

Where the proteasome inhibitor is bortezomib, the amount administered in a single dose can range from 0.7 to 1.3 mg/m² per dose. The amount administered in a single unit dose can be 0.7, 1.0, or 1.7 mg/m² per dose. Where the bortezomib is administered twice weekly for two out of every three weeks, the amount administered can range from 0.7 to 1.3 mg/m² per dose. The method of the invention can also be described in terms of the amount of bortezomib administered per treatment cycle. The per-cycle amount will typically be greater than 2.8, and more usually greater 4.0 mg/m². Typically the per-cycle amount will be at least 5.2 mg/m². Alternatively, the amount of bortezomib administered is at least 2.8 to 5.2 mg/m² per treatment cycle or 4.0 to 5.2 mg/m² per treatment cycle.

As noted above, the frequency of the administration of the unit dose is once weekly or twice weekly. In one embodiment of the method of the invention, the pharmaceutical formulation is administered intravenously twice weekly for 2 weeks every 3 or 4 weeks. In one embodiment, the patient is administered a pre-treatment medication to prevent or ameliorate treatment related toxicities. Illustrative pre-treatment medications are described in the examples below. In one embodiment of the method of the invention, the administration of 17-AAG or 17-AG or a prodrug of either is performed on day 1, 4, 8 and 11 of each cycle, and the cycle time is 3 weeks. 17-AAG will typically be administered by intravenous infusion, infused in a period of at least 30, 60, 90, or 120 minutes. For patients with a body surface area (BSA) greater than 2.4 m², dosing can be calculated in accordance with the methods herein using a maximum BSA of 2.4 m².

In human clinical trials of the method of the invention, the following administration regimens have been employed without reaching dose limiting toxicity (DLT) in any treated patient: 275 mg/m² per single administration of 17-AAG twice weekly for two out of three weeks (Days 1, 4, 8, and 11, with a cycle time of 21 days).

As noted above, after 17-AAG is administered, the major metabolite 17-AG, having anti-cancer activity in its own right, appears in the subject. 17-AAG and 17-AG are thus each, and together, responsible for the therapeutic benefit of the method of the invention. The therapeutically effective dose and dosing regimen of 17-AAG is one that achieves an Area Under Curve (AUCₜₒₜₐₗ) of 17-AAG and/or 17-AG in the subject as described herein. Various therapeutically effective doses and dose regimen are illustrated in the examples below. Therapeutically effective doses and dosing regimen of 17-AAG and/or 17-AG provided by the present invention can also be described in terms of Terminal Half Life (t_{1/2}); Clearance (CL); and/or Volume of Distribution in the elimination phase or steady state (V_{Z} and/or V_{SS}).

The therapeutic benefit from the treatment method of the present invention can be observed in responding subjects as soon as 3, 6, 12, 18 or 24 weeks from the start of treatment. In one embodiment, a therapeutic benefit from the treatment is a reduction in a serum protein, and/or BUN or serum calcium, of the patient. In various embodiments, the reduction is at least 25%; at least 50% to 80%; at least 90%; and 100%. The reduction in serum M protein can be determined, for example, by serum protein electrophoresis or immuno-fixation techniques. The percent reduction is the level of the serum M protein, BUN, or calcium in the patient, measured after a period of treatment and then compared to the level of the serum M protein, BUN, or calcium in the patient measured just prior to treatment. Serum proteins are proteins that, when present in elevated levels in the serum, indicate the subject suffers from MM. Such serum proteins include, but are not limited to, serum M protein (also known as serum M paraprotein), β-2 microglobulin, light chain, and total protein.

Other therapeutic benefits that can be achieved via the present invention include one or more of the following: decrease in BM plasmaocytosis, alleviation of anemia, alleviation of renal failure, alleviation of hypercalcemia, and/or reduction/alleviation of lytic bone lesions. Another therapeutic benefit is an improvement of the KPS of the patient by 10% or more, 20% or more, 30% or more, 40% or more, or 50% or more. Another therapeutic benefit is an improvement of the ECOG of the patient by 1 or more, 2 or more, or 3 or more.

Ideally, practice of the present invention does not result in unmanageable hematologic or non-hematologic toxicity. Hematologic toxicities to be avoided include: Grade 4 neutropenia, Grade 4 thrombocytopenia, and/or Grade 4 anemia. Non-hematologic toxicities include: any ≧Grade 3 non-hematologic toxicity (except Grade injection site reaction, alopecia, anorexia, and/or fatigue), nausea, diarrhea and/or vomiting ≧Grade 3 (despite use of maximal medical intervention and/or prophylaxis), and/or treatment delay of more than 4 weeks due to prolonged recovery from a drug related toxicity. Those of skill in the art will recognize that various toxicities may occur in a cancer patient; the method of the present invention provides the benefit of reduced or elimination of the occurrence of such toxicities.

Where the pharmaceutical formulation comprises an additional compound that might cause an anaphylactic reaction (like Cremophor®), additional medications can be administered to prevent or reduce the anaphylactic reaction, such as (a) loratidine or diphenhydramine, (b) famotidine, and (c) methylprednisone or dexamethasone.

The present invention also provides, in various embodiments, methods for treating MM by administering 17-AAG or 17-AG, or a prodrug of either, in combination with a proteasome inhibitor and a third anti-cancer compound, which can be, for example, Thalomid®, Aredia®, and Zometa® or Revlimid® (lenalidomide). The other anti-cancer drug or agent can be administered in unit doses and dosing regimen currently employed in the art.

Importantly, the present invention can be used to treat patients with MM who have failed at least one prior anti-cancer therapy regimen, that is, have refractory or relapsed refractory MM. These prior anti-cancer therapies include, but are not limited to, monotherapy (single agent therapy) or combination therapies of the following treatments and anti-cancer agents: chemotherapy, stem cell transplantation, Thalomid®, Velcade®, and Revlimid®. Chemotherapy includes treatment with a combination melphalan and prednisone (MP), VAD, or an alkylating agent alone or in combination with other agent(s), such as cyclophosphamide plus etoposide or combinations of etoposide, dexamethasone, doxorubicin.

Diagnostic and laboratory methods and tests that may be of benefit in practice of the present invention are well known to one of ordinary skill in the art. See, for example, Pagana and Pagana, Mosby's Manual of Diagnostic and Laboratory Tests, 2d Ed., Mosby-Year Book, 2002 and Jacobs & DeMott Laboratory Test Handbook, 5^{th} Ed., Jacobs et al. (eds), Lexi-Comp, Inc., 2001 (each incorporated herein by reference). Free kappa and free lambda light chain concentrations in serum can be measured using Freelite™ (The Binding Site Inc., Birmingham, United Kingdom).

An active pharmaceutical ingredient (“API,” 17-AAG, 17-AG, prodrug, proteasome inhibitor, other anti-cancer compound, etc.) useful in the method of the present invention can be formulated for administration orally or intravenously, in a suitable solid or liquid form. See Gennaro, ed., Remington: The Science and Practice of Pharmacy, 20th Ed. (Lippincott Williams & Wilkins 2003), incorporated herein by reference. The API can be compounded, for example, with a non-toxic, pharmaceutically acceptable carrier or excipient for solutions, emulsions, suspensions, or any other form suitable for enteral or parenteral administration. Pharmaceutically acceptable carriers include water and other carriers suitable for use in manufacturing preparations in liquefied form. In addition, auxiliary stabilizing, thickening, and coloring agents may be used.

An API useful in the method of the invention may be formulated as microcapsules, nanoparticles, or nanosuspensions. General protocols for such formulations are described, for example, in Microcapsules and Nanoparticles in Medicine and Pharmacy by Max Donbrow, ed., CRC Press (1992) and in Bosch et al. (1996), De Castro (1996), and Bagchi et al. (1997). By increasing the ratio of surface area to volume, these formulations are especially suitable for the delivery of 17-AAG or another relatively insoluble API.

17-AAG can be formulated in an emulsion with vitamin E or a PEGylated derivative thereof. Generic approaches to formulations with such excipients are described in Quay et al. (1998) and Lambert et al. (2000). The 17-AAG can be dissolved in an aqueous solution containing ethanol (preferably less than 1% w/v). Vitamin E or a PEGylated-vitamin E is added. The ethanol is then removed to form a pre-emulsion that can be formulated for intravenous or oral routes of administration.

Another method for preparing a pharmaceutical formulation useful in the present method involves encapsulating 17-AAG or other API in liposomes. Methods for forming liposomes as drug delivery vehicles are well known in the art. Suitable protocols adaptable for the present invention include those described by Boni et al. (1997), Straubinder et al. (1995), and Rahman et al. (1995) for paclitaxel and by Sonntag et al. (2001) for epothilone, mutatis mutandis. Of the various lipids that may be used in such formulations, phosphatidylcholine and polyethyleneglycol-derivatized distearyl phosphatidyl-ethanoloamine are noteworthy.

The amount of 17-AAG or other API that may be combined with the carrier materials to produce a single or unit dosage form will vary depending upon the subject treated and the particular mode of administration. For example, a formulation for intravenous use comprises an amount of 17-AAG ranging from about 1 mg/mL to about 25 mg/mL, preferably from about 5 mg/mL, and more preferably about 10 mg/mL. Intravenous formulations are typically diluted between about 2 fold and about 30 fold with water for injection (WFI), normal saline, or 5% dextrose solution prior to use. In many instances, the dilution is between about 5 and about 10 fold.

In one embodiment of the method of the invention, 17-AAG is formulated as a pharmaceutical solution formulation comprising 17-AAG dissolved in a vehicle comprising (i) a first component that is ethanol; (ii) a second component that is a polyethoxylated castor oil; and (iii) a third component selected propylene glycol, PEG 300, PEG 400, glycerol, and combinations thereof, as disclosed in Zhong et al. (2005).

Another formulation of 17-AAG that may be used is one based on dimethylsulfoxide (“DMSO”) and egg lecithin (egg phospholipids), as taught in Tabibi et al. (2004). However, because of certain characteristics of DMSO (odor, patient adverse reactions), such formulations are less preferred than the DMSO-free ones taught herein.

Other formulations for 17-AAG that may be employed in the method of the invention are described in Ulm et al. (2003), Ulm et al. (2004), Mansfield et al. (2006), Desai et al. (2006), and Isaacs et al. (2006).

In another embodiment, the pharmaceutical formulation can be diluted 1:7 prior to administration with sterile WFI, USP (one part undiluted drug product to 6 parts sterile WFI). Dilution is performed under controlled, aseptic conditions. The final diluted drug product concentration is, using 17-AAG as an example, at least 1.00 mg/mL, such as approximately 1.43, approximately 2.00 or approximately 10.00 mg/mL.

Depending on the BSA and the assigned dose for the subject, the dose of 17-AAG or other API will require different volumes of drug product to be added to the admixture bag. An overfill can be calculated and employed to account for loss in the administration set. Preferably, the pharmaceutical formulation, with the diluted drug product, is pH neutral, and the solution is hypertonic at approximately 600 mOsm. The pharmaceutical formulation can be stored at −20° C., with protection from light. Drug product is allowed to come to room temperature prior to admixture and then is mixed is by gentle inversion. After dilution, the drug product should stable for up to about 10 hours at room temperature (at a dilution of 1:7).

The present invention, having been described in summary fashion and in detail above is illustrated in the following Examples.

### EXAMPLE 1

### Treatment of Patients with Multiple Myeloma with 17-AAG in Combination with Bortezomib

The method of the invention was tested in an open-label, dose escalating clinical trial. The trial was designed to establish the MTD of 17-AAG administered by IV infusion over 60 minutes, co-administered with bortezomib, on Days 1, 4, 8, and 11 of a dosing cycle lasting 3 weeks. The dose-escalating component of this trial began with bortezomib administered at approximately 50% of its recommended dose and the starting dose of 17-AAG set at slightly less than 50% of its single-agent dose using a previous formulation (100 mg/m²). Doses of each agent were then escalated until the MTD for the combination could be ascertained.

Disease response evaluations were performed following every two cycles of treatment (approximately every 6 weeks). The determination of anti-tumor efficacy in stable or responding patients was based on objective tumor assessments made according to a standardized myeloma response assessment system.

All baseline imaging-based tumor assessments were performed within 28 days prior to the start of treatment and reevaluated every 6 weeks (approximately every two cycles) thereafter. All patients with responding tumors (CR or PR) were examined to confirm the response 6 weeks after the first documentation of response. Response criteria used were according to guidelines of Bladé et al. (1998).

Pharmacokinetic (PK) and pharmacodynamic (PD) sampling was obtained during the first treatment cycle only. In the event of drug-related serious adverse events (SAEs) and/or Grade 4 toxicities, additional PK samples were to be collected.

MM patients enrolled in this study were those who had failed at least two prior anti-cancer therapy regimens. The enrollment criteria were: (1) patients were at least 18 years old; (2) had a KPS performance status of ≧70%; (3) had histologic evidence of MM but did not necessarily have measurable disease, although disease had to have been assessed within 28 days prior to treatment initiation; (4) were, with respect to all adverse events of any prior chemotherapy, surgery, or radiotherapy, resolved to NCI CTCAE (v. 3.0) Grade≦2; and (5) had the following laboratory results within 10 days of 17-AAG administration: hemoglobin≧8 g/dL, absolute neutrophils count≦1.5×10⁹/L, platelet count≧75×10⁹/L, serum bilirubin≦2× upper limit of normal (ULN), AST≦2.5 ULN, and serum creatinine≦2× ULN.

Patients were graded according to the KPS Performance Status scale and criteria as described in Table 1. Patients were excluded from the study if they had a condition such as pre-existing neuropathy, pregnancy, breast-feeding, recent chemotherapy, and so forth. To be eligible for enrollment, patients also had to meet certain hematologic conditions.

17-AAG is highly protein bound in plasma (approximately 95% in in vitro assays using human blood); however, the plasma protein to which the drug binds and the affinity of binding are not known. Patients who are receiving agents that are known to be highly protein bound were subjected to close clinical monitoring while enrolled in the trial. In vitro studies implicate the involvement of cytochrome P450 enzymes in the metabolism of 17-AAG. No formal drug-drug interaction studies have been performed with 17-AAG and drugs that are substrates, inhibitors, or inducers of cytochrome P450-3A4. While there is no contraindication to the concomitant use of any medication with 17-AAG, 17-AAG was used with caution in combination with drugs that are also highly protein bound (e.g. warfarin) and drugs that are a substrate, inhibitor, or inducer of cytochrome P450-3A4. Hormonal contraceptives were not used in women of childbearing potential enrolled in the trial. No other investigational agents are permitted during the entire duration of the study (from 3 weeks before the first administration until the end to treatment evaluation).

PK assessments included the following tests. Blood samples for determination of plasma concentrations of the parent compound and its primary metabolite were collected following the first and fourth 17-AAG administration only (Day 1 and 11). The total number of PK samples collected was approximately 115 mL of whole blood (7-8 tablespoons). If a patient experienced a potentially drug-related SAE, additional PK samples were collected. Blood was drawn from the contralateral arm to the infusion site using an indwelling catheter to avoid multiple needle sticks. For the 17-AAG samples, 5 mL of blood was drawn into a vacuum tube containing heparin as anti-coagulant. The blood tube was inverted several times and the tube placed in wet ice immediately pending separation of the plasma. If a catheter was used for blood collection, the fluid in the catheter was completely withdrawn prior to each sample collection and discarded. Plasma samples were kept on wet ice during collection and centrifugation. Plasma samples were split into two cryovials prior to freezing at −70° C. Plasma concentrations of 17-AAG and its primary metabolite 17-AG were measured by a validated LC/MS method. (Egorin et al., 1998.)

PD assessment included the following tests. (1) Clinical correlates: the occurrence of specific toxicities of interest (e.g., severity, duration and reversibility) was compared to PK parameters (e.g., clearance, exposure, elimination half-life, maximal plasma concentration, and time above a target plasma concentration). These included hepatotoxicity and gastrointestinal toxicities. (2) Multiple myeloma cells: (i) surface expression of IL-6R, insulin-like growth factor receptor-1 (IGF-1R) in MM cells; (ii) total expression of phospho-AKT, Akt, Hsp90 and Hsp70 in MM cells; and (iii) gene expression profiling to identify other potential bio-markers for drug sensitivity versus resistance. MM cells were purified from bone marrow (BM) aspirates performed at baseline (up to 3 weeks prior to first study drug administration), 3-4 hours following the fourth infusion of 17-AAG and bortezomib (Day 11), and after the end of treatment (or at time of progressive disease). MM cells were purified from the BM aspirates based upon CD138 expression using magnetic bead technology and confirmed by flow cytometric analysis to be >95% CD138⁺ MM cells. Flow cytometric analysis assesses IGF-R surface expression using fluorescein isothyocynate (FITC)-conjugated anti-human IGF-R monoclonal antibody (R&D Systems, Minneapolis, Minn.). Immunoblotting analyses evaluated the total levels of phospho-AKT, AKT, Hsp90 and Hsp70. (3) Peripheral blood mononuclear cells: PBMCs were obtained (pre-therapy and 4 hours following the bortezomib intravenous bolus on Days 1 and 11) and examined for change in Hsp70, Hsp90, and others as indicated via Western Blot. For PBMC isolation, blood was collected into preservative-free heparin and PBMCs isolated by Ficoll-Paque density gradient centrifugation. (4) The percentage inhibition of proteasome function (evaluated by measurement of 20S proteasome activity) was performed, according to the method of Lightcap et al (2000). Whole blood lysates were obtained prior to the infusion, 1, 4 and 24 hours following the IV bolus of bortezomib on Days 1 and 11. (5) Plasma: whole blood (8 cc per timepoint) was collected into EDTA-containing tubes.

The end-of-treatment assessment was conducted as follows. The planned treatment period was 24 weeks (8 cycles). Patients were treated in the absence of progressive disease or unacceptable treatment-associated toxicities. All patients who received at least one dose of the study drug and discontinued treatment for any reason (except death) had the end of treatment assessment performed. The assessment occurred up to 28 days following the last receipt of 17-AAG and included a physical examination, with body weight and vital signs measurements, documentation of KPS Performance Status, hematology, coagulation and chemistry/electrolyte determinations, urinalysis, assessment of the patient's current medications and ongoing clinical adverse events (if any). Tumor assessments (myeloma laboratory tests, assessment of extramedullary disease, BM aspirate, and other radiographic staging, if appropriate) were done at this time only if the previous assessment occurred more than 4 weeks prior to withdrawal.

Bortezomib (obtained commercially) was administered intravenously twice weekly for 2 weeks (on Day 1, 4, 8 and 1) every 3 weeks at escalating doses (calculated mg/m²) administered as a rapid (3-5 second) injection. Bortezomib was administered per its Package Insert (incorporated herein by reference). The starting dose of bortezomib was 0.7 mg/m²; doses were escalated based on observed toxicities. The dose did not escalate beyond its recommended dose for single-agent therapy in this population (1.3 mg/m²).

17-AAG was administered intravenously twice weekly for 2 weeks (on Day 1, 4, 8 and 11) every 3 weeks at escalating doses (calculated mg/m²) infused over 60 minutes after pre-medication. For patients with a body surface area (BSA) greater than 2.4 m 2, dosing was calculated using a maximum BSA of 2.4 m².

The preparation and administration of 17-AAG was as follows. 17-AAG was dissolved in 30% propylene glycol, 20% Cremophor® EL, and 50% ethanol to a concentration of 10 mg/mL in the vial. Drug product was available in 20 mL type 1 clear glass vials with a 20 mm finish (containing 200 mg/vial). The vials were closed with gray 20 mm Teflon coated serum stoppers and white 20 mm flip-off white lacquered flip tops. It was diluted 1:7 prior to administration with sterile WFI, USP (one part undiluted drug product to 6 parts sterile WFI). Dilution was performed under controlled, aseptic conditions. Final diluted drug product had a concentration of approximately 1.43 mg/mL. 17-AAG was prepared either using glass vacuum containers or compatible non-PVC, non-DEHP (di(2-ethylhexyl)-phthalate) IV admixture bags. Both systems require non-PVC, non-DEHP containing administration sets and either an in-line 0.22 μm filter or use of an extension set containing such a filter. Due to the light sensitivity of 17-AAG, protection from light is advised.

For glass collection units, examples of compatible supplies includes Baxter 1A8502 (or equivalent), using a Baxter 2C1106 or equivalent IV administration set with extension set with 0.22 μm air eliminating filter (Baxter 1C8363 or equivalent). For non-PVC, non-DEHP admixture bags, compatible admixture bags may be empty or pre-filled with 250 cc WFI. Examples of compatible admixture bags include Excel (250 cc WFI; made from polyolefin).

Depending on the body surface area and the assigned dose for individual patients, the dose of 17-AAG required different volumes of drug product to be added to the admixture bag. An overfill was calculated to account for any loss in the administration set.

As noted above, 17-AAG was administered intravenously twice weekly for 2 weeks out of every 3 weeks. The total dose delivered is rounded to the nearest milligram.

Pre-medication treatments were conducted as follows. All patients were pre-medicated prior to each infusion of 17-AAG. An appropriate pre-medication regimen was used for each patient based upon past history of potential Cremophor®-induced hypersensitivity reactions and the type and severity of the hypersensitivity reaction observed following treatment with 17-AAG. The standard premedication regimen was to pre-medicate with loratidine 10 mg p.o., famotidine 20 mg p.o., and either methylprednisolone 40-80 mg IV or dexamethasone 10-20 mg IV 30 minutes prior to infusion of 17-AAG. Choice of antihistamine and corticosteroid, route of administration, doses prior to 17-AAG infusion was at the investigator's discretion, but was similar to prophylaxis for other Cremophor®-containing products (such as Taxol®, paclitaxel). Doses of corticosteroid were lowered if the patient is receiving concomitant prednisone. The high dose premedication regimen was to pre-medicate with diphenhydramine 50 mg IV, famotidine 20 mg IV and either methylprednisolone 80 mg IV or dexamethasone 20 mg IV (or split as oral doses of 10 mg each 6 and 12 hours prior to the infusion), at least 30 minutes prior to the infusion of 17-AAG. The choice of antihistamine and corticosteroid was at the investigator's discretion.

The doses and schedule of study drugs was as follows. Patients received therapy on Days 1, 4, 8 and 11 in 3-week cycles. Therapy consisted of bortezomib administered as an intravenous rapid (3-5 second) bolus, followed by 17-AAG administered via intravenous infusion (IV) over 60 minutes. The infusion of 17-AAG was elongated to 90 or 120 minutes if necessary at the higher doses due to volume of administration. For the initial administration, all patients were administered with 17-AAG with bortezomib, except for patients who had failed bortezomib therapy immediately prior to study entry.

The initial patient cohort received bortezomib at dose of 0.7 mg/m², followed by an intravenous infusion of 17-AAG at dose of 100 mg/m² (cohort 1). Subsequent patient cohorts were enrolled per the escalation scheme as follows: bortezomib at a dose of 1.0 Mg/m² and 17-AAG at a dose of 100 Mg/m² (cohort 2), bortezomib at a dose of 1.0 mg/m² and 17-AAG at a dose of 150 mg/m² (cohort 3), bortezomib at a dose of 1.3 mg/m² and 17-AAG at a dose of 150 mg/M² (cohort 4), bortezomib at a dose of 1.3 mg/m² and 17-AAG at a dose of 220 mg/m² (cohort 5), bortezomib at a dose of 1.3 mg/m² and 17-AAG at a dose of 275 mg/m² (cohort 6), and bortezomib at a dose of 1.3 mg/m² and 17-AAG at a dose of 340 mg/m² (cohort 7).

Three patients were assigned to each cohort. If no DLT is observed in a cohort evaluable for a dose escalating decision (“evaluable” is defined here as having received four treatments in a 3-week period or having withdrawn due to drug-related toxicity), then the next dose level was evaluated. If one or more patients experience a DLT, then the cohort was increased to six evaluable patients. If two or more of six evaluable patients entered in a cohort experienced a DLT then the MTD had been exceeded; all further accrual would be at the previous dose level. If no more than one of the six patients experienced a DLT then the next dose level was evaluated. Once the MTD was defined an additional number of patients were enrolled to arrive at a cumulative total of 12 patients at the MTD dose level. Eighteen patients were treated in accordance with this protocol.

Of the eighteen patients, 9 were male and 9 were female. Their median age was 63 years old (having a range of 44 to 81 years old). Their subtype were 72% were IgG and 28% were IgA. The KPS median was 90 (having a range of 70 to 100). The number of prior chemotherapy was 4 (having a range of 2 to 16). Prior chemotherapy included inter alia bortezomib, thalidomide, VAD/VdD, melphalan, and lenalidomide. The number of patients with prior transplants was 12 (67%). The number of patients with extramedullary disease was 4 (22%). The median baseline β-2 microglobulin was 3.7 (having a range of 1.4 to 9.7). The median time since diagnosis of MM was 61 months (having a range of 14 to 238 months).

Three patients (cohort 1; Patients 101-103) were first administered with 0.7 mg/m² of bortezomib (infused as a rapid 3-5 seconds intravenous push), and then administered a 100 mg/m² dose of 17-AAG (one hour intravenous infusion), twice weekly for every 2 out of 3 weeks (Days 1 and 11 of the first treatment cycle). Patients underwent a mean of 3.3 cycles of treatment. DLT was not observed in any the three patients. Of the three patients, after treatment, stable disease was observed in one patient who underwent 5 cycles of treatment (33% of all patients treated at this dose level), and progressive disease was observed in two patients (67% of all patients treated at this dose level).

Three patients (cohort 2; Patients 201, 203 and 204) were first administered with 1.0 mg/m² of bortezomib (infused as a rapid 3-5 seconds intravenous push), and then administered a 100 mg/m² dose of 17-AAG (one hour intravenous infusion), twice weekly for every 2 out of 3 weeks (Days 1 and 11 of the first treatment cycle). Patients underwent a mean of 11.3 cycles of treatment. DLT was not observed in any the three patients. Of the three patients, after treatment, treatment resulted in MR for all three patients (100% of all patients treated at this dose level). One of the three patients was a bortezomib naïve patient. Two patients underwent at least 9 cycles of treatment. One patient underwent 9 cycles of treatment this dose level and was then escalated to dose level 3 for the tenth cycle upon which a MR was observed. This patient has undergone at least 13 treatment cycles.

Eight patients (cohort 3; Patients 301-308) were first administered with 1.0 mg/m² of bortezomib (infused as a rapid 3-5 seconds intravenous push), and then administered a 150 mg/m² dose of 17-AAG (one hour intravenous infusion), twice weekly for every 2 out of 3 weeks (Days 1 and 11 of the first treatment cycle); with the following exceptions: three had infusions that were 1.6 to 2 hours long (patients 303, 305 and 306). Patients underwent a mean of 4.3 cycles of treatment (and treatment is still ongoing). By 6.0 or more cycles of treatment, one patient was identified with a Grade 4 hepatotoxicity with a 1.4 cm plasmacytoma in the liver, amyloidosis in the liver and heart, and an increase of ALT/AST. There was one death caused an unrelated cause (cardiac amyloidosis). nCR was observed in two patients. One of the two patients was a bortezomib naïve patient. MR was observed in one patient. SD was observed in two patients. Of the two patients, one was bortezomib naïve. One patient was observed having PD. Two patients were not evaluable.

Four patients (cohort 4; Patients 401-404) were first administered with 1.3 mg/m² of bortezomib (infused as a rapid 3-5 seconds intravenous push), and then administered a 150 mg/m² dose of 17-AAG (one hour intravenous infusion), twice weekly for every 2 out of 3 weeks (Days 1 and 11 of the first treatment cycle). Patients underwent a mean of 4.5 cycles of treatment (and treatment is still ongoing). One patient was identified with a Grade 3 pancreatitis (the assessment is still pending). Three patients were observed to have MR. Of the three patients, one was bortezomib naïve.

The other drug-related toxicities observed in these patients included Grade 1-2 elevated transaminases, nausea, fatigue, diarrhea, anemia, myalgias, rash, and infusional reactions, and thrombocytopenia.

Blood was collected for PK analysis as follows for plasma drug concentration analysis: pre-dose, 30 minutes intra-infusion, just before the end-of-infusion (EOI), 5, 15, 30 mins and 1, 2, 4, 8 and 24 hours post infusion. For every patient (except Patient #301) neither the parent nor the metabolite were detectable by Day 4 and repeat PK on Day 11 of each 3 week cycle.

The plasma profiles showed a rapid elimination of parent drug (17-AAG) and a much slower elimination of the metabolite (17-AG).

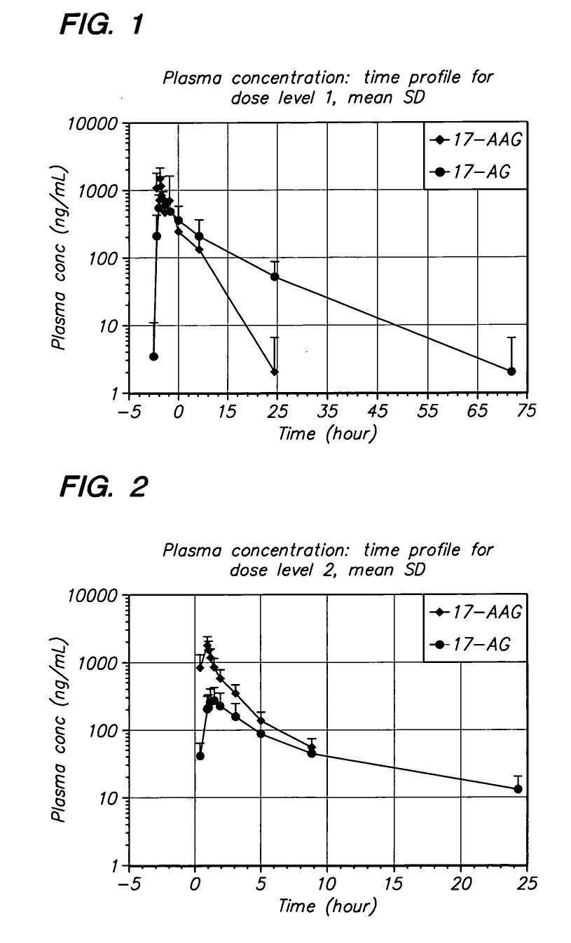

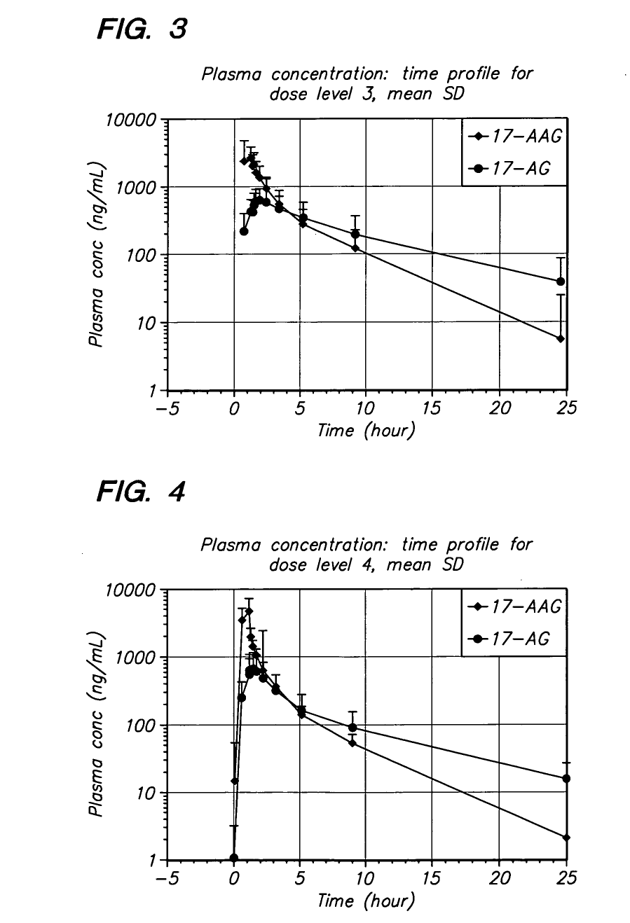

All six patients of cohorts 1 and 2 received 100 mg/m² 17-AAG. Metabolite was detected in the 72 hour sample in one of the six patients (patient 103) at 10.2 ng/mL. FIGS. 1 and 2 show the plasma concentration profile for 17-AAG and 17-AG for these two dose levels.

Following the end of the infusion, the plasma profile of 17-AAG and 17-AG were similar for the Day 1 and Day 11 administrations. Allowing for the fact that on Day 11 the end-of-infusion sample was not collected, the curves were probably indistinguishable. There was also metabolite concentration in the pre-dose plasma on Day 11.

Plasma concentration versus time results were analyzed using non-compartmental methods to determine the pharmacokinetics of 17-AAG and 17-AG using Kinetica version 4.3 software (Innaphase, Champs sur Marne, France). Mean patient results and statistical summaries are presented in Tables 2 (17-AAG) and 3 (17-AG).

TABLE 2
PK Parameters for 17-AAG

| Patient | Dose | Infusion | Cₘₐₓ | Tₘₐₓ | AUCₗₐₛₜ |
|---|---|---|---|---|---|
| (ID & Day) | (mg) | duration (h) | (ng/mL) | (h) | (ng/mL * h) |
| Cohorts 1 & 2 | | | | | |
| Mean | 186 | 1.1 | 1,788 | 1 | 3,580 |
| SD | 31.01 | 0.28 | 596 | 0.68 | 1,315.38 |
| CV % | 16.67 | 25.13 | 33.33 | 53.49 | 36.74 |
| Min | 132 | 1 | 755 | 0.5 | 2,231.7 |
| Max | 215 | 1.92 | 2,620 | 3 | 7,248.8 |
| Median | 200 | 1 | 1,740 | 1 | 3,347.9 |
| Cohorts 3 & 4 | | | | | |
| Mean | 264 | 1 | 3,472 | 1 | 5,774 |
| SD | 29 | 0 | 1,893 | 0 | 3,192 |
| CV % | 11.09 | 29.93 | 54.54 | 40.41 | 55.28 |
| Min | 219 | 0.97 | 1,830 | 0.5 | 3,152 |
| Max | 309 | 2.08 | 9,540 | 2.17 | 17,650 |
| Median | 268 | 1 | 2,740.5 | 0.97 | 4,871 |

| Patient | AUCₑₓₜᵣₐ | AUCₜₒₜₐₗ | AUCₑₓₜ | L_{z} |
|---|---|---|---|---|
| (ID & Day) | (ng/mL * h) | ng/mL * h | (%) | (l/h) |
| Cohorts 1 & 2 | | | | |
| Mean | 225 | 3,805 | 6 | 0.30376 |
| SD | 153.35 | 1,438.05 | 2.40 | 0.07886 |
| CV % | 68.18 | 37.79 | 41.89 | 25.96 |
| Min | 87.9 | 2,380.5 | 2.54 | 0.13432 |
| Max | 608.6 | 7,857.4 | 10.80 | 0.42043 |
| Median | 165.9 | 3,513.8 | 6.10 | 0.30254 |
| Cohorts 3 & 4 | | | | |
| Mean | 256 | 6,030 | 5 | 0.3127 |
| SD | 162 | 3,253 | 3 | 0.0744 |
| CV % | 63.45 | 53.95 | 59.18 | 23.78 |
| Min | 54 | 3,294 | 0.9 | 0.1208 |
| Max | 595 | 18,246 | 10.5 | 0.4276 |
| Median | 199 | 5,071 | 3.8 | 0.3160 |

| Patient | BSA | t_{1/2} | MRT | Clearance | Clearance |
|---|---|---|---|---|---|
| (ID & Day) | (m²) | (h) | (h) | (L/h) | (L/h/m²) |
| Cohorts 1 & 2 | | | | | |
| Mean | 1.86 | 2.49 | 3.32 | 52.76 | 28.63 |
| SD | 0.31 | 0.97 | 1.06 | 15.44 | 7.57 |
| CV % | 16.67 | 38.74 | 31.91 | 29.26 | 26.44 |
| Min | 1.32 | 1.72 | 2.37 | 26.73 | 12.73 |
| Max | 2.15 | 5.16 | 6.04 | 84.33 | 42.01 |
| Median | 2 | 2.29 | 2.887 | 55.45 | 28.46 |
| Cohorts 3 & 4 | | | | | |
| Mean | 1.76 | 2.40 | 2.89 | 51.00 | 28.94 |
| SD | 0.19 | 0.88 | 1.02 | 16.84 | 9.12 |
| CV % | 11.1 | 36.7 | 35.2 | 33.0 | 31.5 |
| Min | 1.5 | 1.6 | 1.5 | 13.2 | 8.2 |
| Max | 2.1 | 5.7 | 6.0 | 75.3 | 45.5 |
| Median | 1.8 | 2.2 | 2.9 | 50.6 | 29.6 |

| Patient | V_{z} | V_{z} | Vₛₛ | Vₛₛ |
|---|---|---|---|---|
| (ID & Day) | (L) | (L/m²) | (L) | (L/m²) |
| Cohorts 1 & 2 | | | | |
| Mean | 192.65 | 104.58 | 174.19 | 93.88 |
| SD | 96.18 | 49.81 | 74.56 | 35.69 |
| CV % | 49.92 | 47.63 | 42.81 | 38.01 |
| Min | 72.61 | 34.58 | 96.59 | 48.42 |
| Max | 430.47 | 215.23 | 349.09 | 174.55 |
| Median | 188.14 | 99.17 | 159.32 | 79.65 |
| Cohorts 3 & 4 | | | | |
| Mean | 165.99 | 94.79 | 143.07 | 80.89 |
| SD | 49.35 | 28.37 | 54.12 | 29.27 |
| CV % | 29.7 | 29.9 | 37.8 | 36.2 |
| Min | 56.8 | 31.5 | 35.8 | 19.9 |
| Max | 244.8 | 149.3 | 221.9 | 135.3 |
| Median | 172.5 | 93.7 | 151.4 | 84.3 |

TABLE 3
PK Parameters for 17-AG

| Patient | Dose | Cₘₐₓ | Tₘₐₓ | t_{1/2} | AUCₗₐₛₜ |
|---|---|---|---|---|---|
| (ID & Day) | (mg/m²) | (ng/mL) | (h) | (h) | (ng/mL * h) |
| Cohorts 1 & 2 | | | | | |
| Mean | 100 | 523 | 1.42 | 7.51 | 3,478.2 |
| SD | 0 | 412 | 0.32 | 2.24 | 3,547.1 |
| CV % | 0 | 78.8 | 22.5 | 29.9 | 102.0 |
| Min | 100 | 146 | 1 | 3.77 | 628.5 |
| Max | 100 | 1,510 | 2.167 | 11.89 | 10,592.6 |
| Median | 100 | 389 | 1.417 | 7.05 | 1,783.5 |
| Cohorts 3 & 4 | | | | | |
| Mean | 150 | 669 | 1.6 | 6.0 | 3,984 |
| SD | 0 | 303 | 0.5 | 1.2 | 2,833 |
| CV % | 0.00 | 45.31 | 32.39 | 20.30 | 71.1 |
| Min | 150 | 237 | 1.08 | 4.58 | 934 |
| Max | 150 | 1,360 | 3 | 9.06 | 13,720 |
| Median | 150 | 685 | 1.45 | 5.77 | 3,080 |

| Patient | AUCₑₓₜᵣₐ | AUCₜₒₜₐₗ | AUCₑₓₜ | L_{z} |
|---|---|---|---|---|
| (ID & Day) | (ng/mL * h) | ng/mL * h | (%) | (l/h) |
| Cohorts 1 & 2 | | | | |
| Mean | 229.5 | 3,707.6 | 8.1 | 0.1007 |
| SD | 320.7 | 3,690.0 | 5.9 | 0.0337 |
| CV % | 139.8 | 99.5 | 71.9 | 33.4 |
| Min | 72.3 | 803.8 | 1.3 | 0.0583 |
| Max | 1,182.0 | 10,730.4 | 21.8 | 0.1841 |
| Median | 122.5 | 1,861.4 | 6.9 | 0.0984 |
| Cohorts 3 & 4 | | | | |
| Mean | 299 | 4,284 | 5.5 | 0.1190 |
| SD | 535 | 3,316 | 3.1 | 0.0209 |
| CV % | 178.7 | 77.4 | 56.3 | 17.55 |
| Min | 46 | 980 | 2.2 | 0.0765 |
| Max | 2,604 | 16,323 | 16.0 | 0.1512 |
| Median | 160 | 3,253 | 5.0 | 0.1200 |

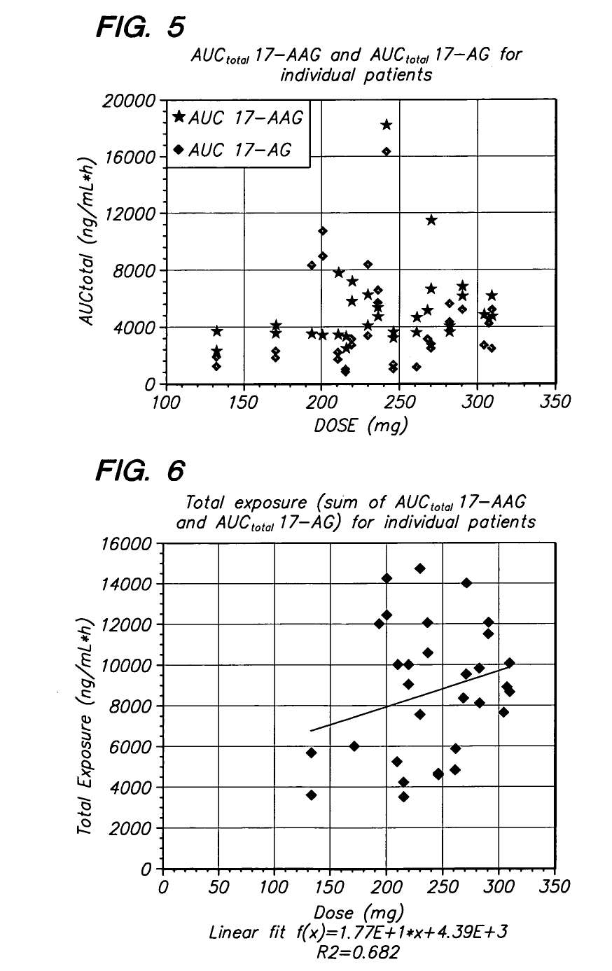

Statistical analysis of the data in Tables 2 and 3 show that the average ratio of AUCₜₒₜₐₗ for the 17-AG to AUCₜₒₜₐₗ for the parent drug 17-AAG was 82.5±90.5%. The average combine exposure (17-AAG plus 17-AG) was 7,513±3,891 ng/mL*h for a dose of 100 mg/m² and was 10,313±6,076 ng/mL*h for a dose of 150 mg/m². FIG. 5 shows the relative values of the AUCₜₒₜₐₗ for metabolite and parent drug. FIG. 6 shows the total exposure for metabolite and parent drug together. The correlation of dose with total exposure was not very strong, R²=0.682. Terminal elimination half-life for 17-AAG was 2.43±0.9 h and for 17-AG 6.52±1.74 hours. Total systemic clearance 17-AAG was 51.58±16.16 L/h or 28.83±8.51 L/h/m². The distributive volumes for 17-AAG were: VZ=174.88±68.2 L or 98.05±36.41 L/m² and VSS=153.44±62.3 L or 85.25±31.60 L/m².

Based on the results for the first four dose cohorts, bortezomib has no effect on the metabolism of 17-AAG.

Pharmacodynamic Analysis

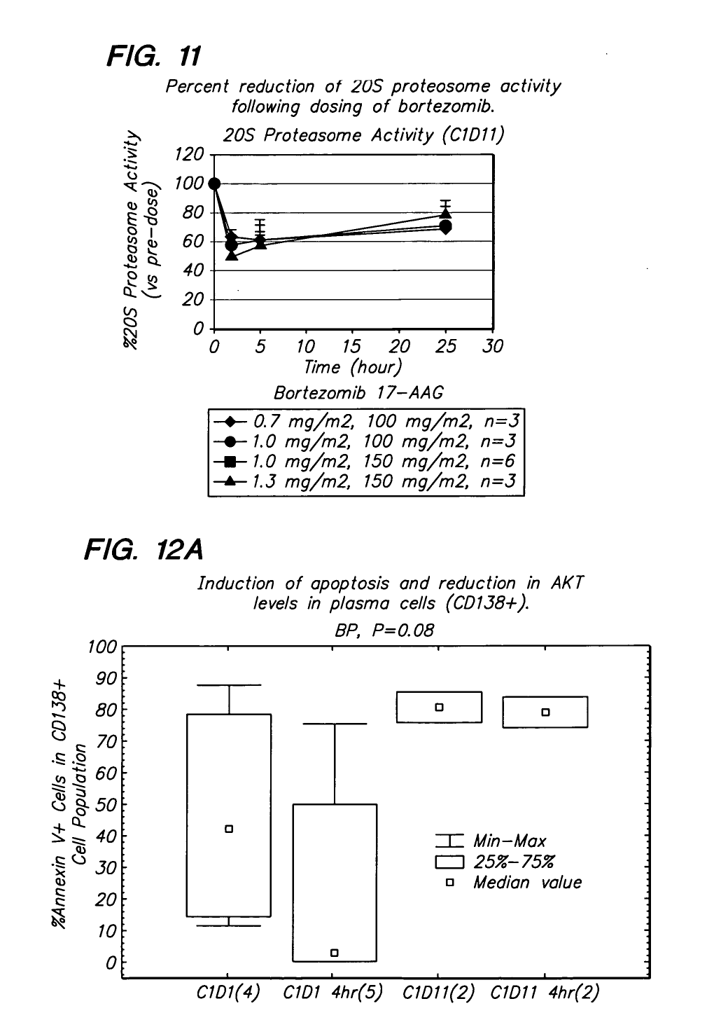

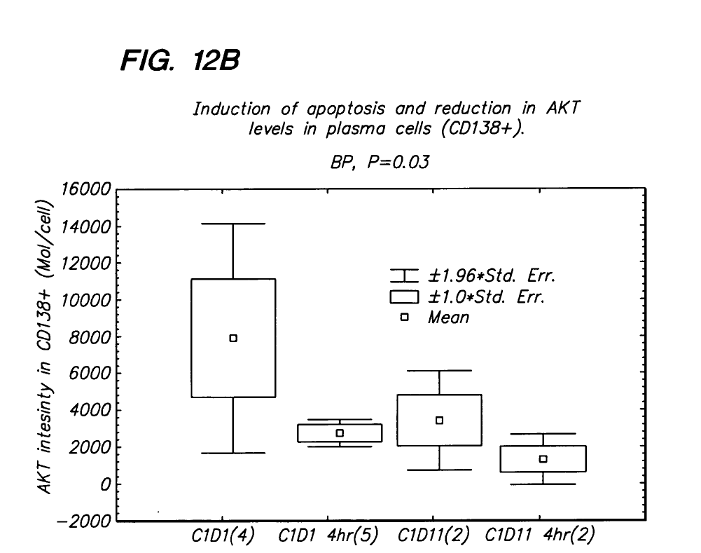

Evaluation of proteasome function showed a 37% to 50% decrease for the 4 doses levels tested at the end-of-infusion (FIG. 11). There was also observed a induction in apoptosis and reduction in AKT levels in plasma cells (CD138⁺) (FIG. 12). AKT is a signaling protein that is up-regulated in myeloma cells on the Ras/Raf/MAPK intracellular pathway critical to myeloma cell growth and progression. Abnormal mitochondrial potential is observed prior to apoptosis of that cell (programmed cell death).

Anti-myeloma activity was observed in bortezomib-naïve and bortezomib-refractory patients. Patients 201, 204, 307 and 308 were observed to have reductions of various proteins in serum and urine.

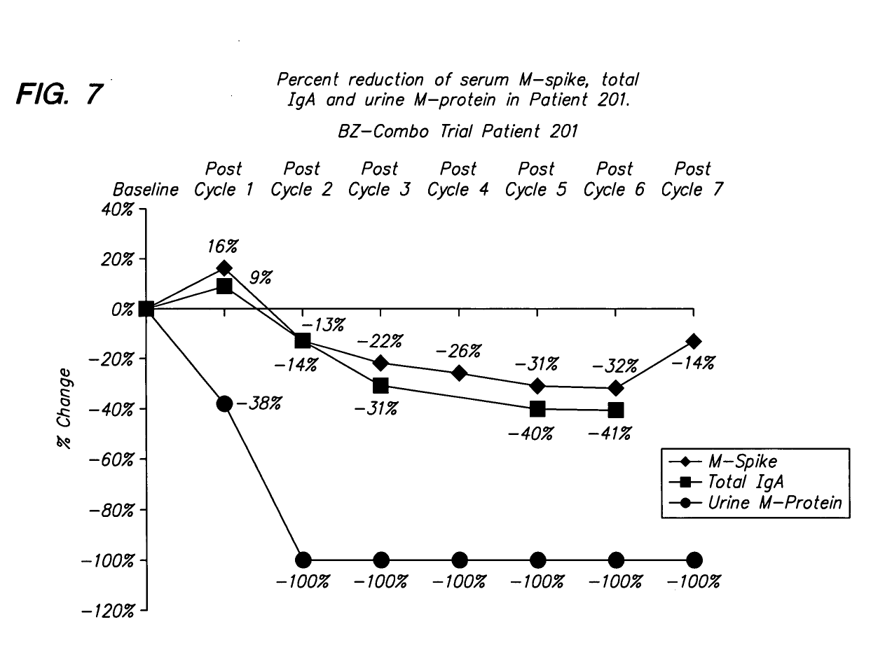

Patient 201 had the prior treatments of VAD, melphalan-corticosteroid weekly, and VAD in combination with Thalidomide®. Disease progression was observed for all these previous treatments. Patient 201 underwent nine cycles of treatment, resulting in an MR. FIG. 7 and Table 4 show the reduction of serum M-spike, total IgA and urine M-protein.

TABLE 4
Patient 201 Serum and Urine Protein Readings

| | M-Spike | Total Ig A | Urine M-Protein |
|---|---|---|---|
| Stage | (g/dL) | (mg/dL) | (mg/24 h) |
| Baseline | 3.94 | 6,620 | 97.2 |
| Post Cycle 1 | 4.57 | 7,230 | 60.2 |
| Post Cycle 2 | 3.39 | 5,770 | 0 |
| Post Cycle 3 | 3.06 | 4,550 | 0 |
| Post Cycle 4 | 2.93 | ND | 0 |
| Post Cycle 5 | 2.73 | 4,000 | 0 |
| Post Cycle 6 | 2.67 | 3,920 | 0 |
| Post Cycle 7 | 3.4 | ND | 0 |

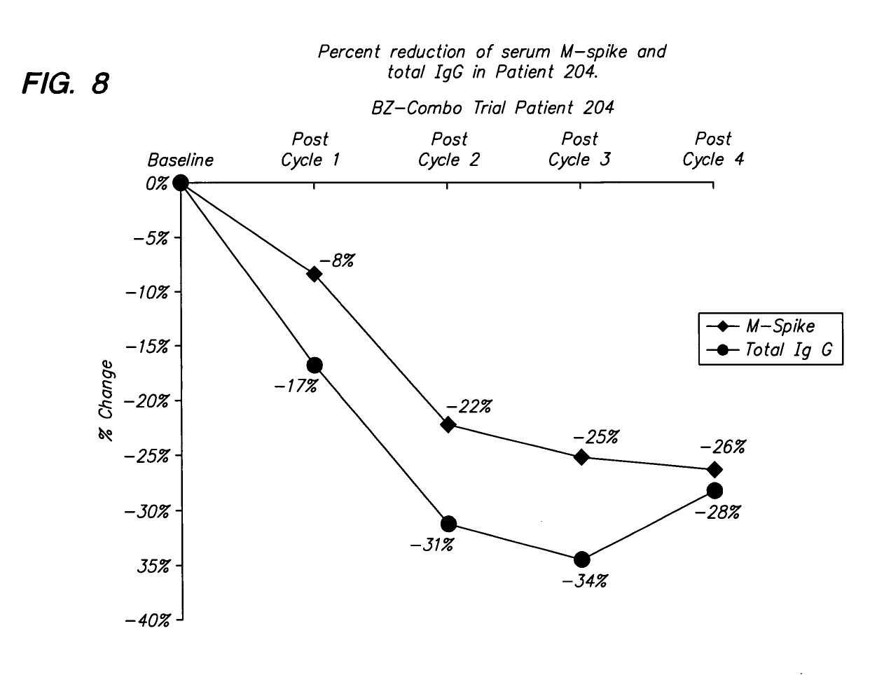

Patient 204 had the prior treatments of MP and Velcade/Doxil/Thalidomide®. Patient 204 has undegone at least six cycles of treatment, resulting in a MR. FIG. 8 and Table 5 show the reduction of serum M-spike and total IgG in Patient 204.

TABLE 5
Patient 204 Serum Protein Readings

| Stage | M-Spike (g/dL) | Total Ig G (mg/mL) |
|---|---|---|
| Baseline | 1.68 | 2,460 |
| Post Cycle 1 | 1.54 | 2,050 |
| Post Cycle 2 | 1.31 | 1,700 |
| Post Cycle 3 | 1.26 | 1,620 |
| Post Cycle 4 | 1.24 | 1,770 |

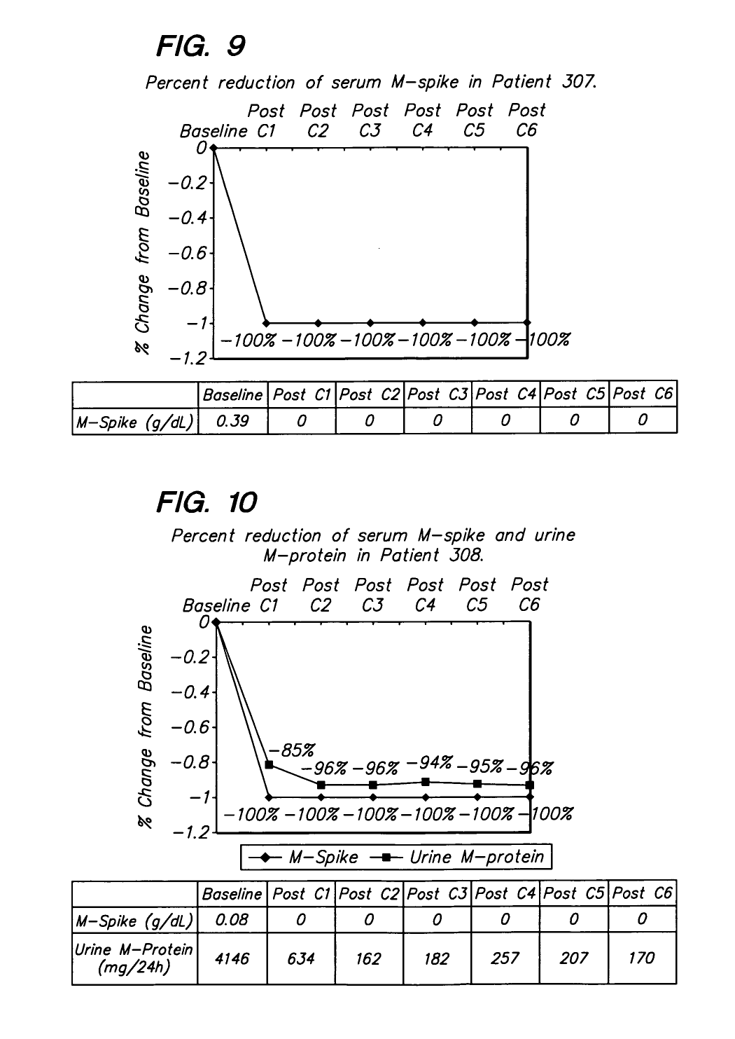

Patient 307 had the prior treatments of VAD, etoposide/cytoxan, interferon, Thalidomide®, and bortezomib/Doxil/Thalidomide®. Patient 307 underwent at least eight cycles of treatment. FIG. 9 shows the reduction of serum M-spike in Patient 307. Treatment for Patient 307 resulted in a nCR.

Patient 308 had the prior treatments of dexamethasone and Thalidomide®/dexamethasone. Patient 308 underwent at least eight cycles of treatment. FIG. 10 shows the reduction of serum M-spike and urine M-protein in Patient 308. Treatment for Patient 308 resulted in a nCR.

Although the present invention has been described in detail with reference to specific embodiments, those of skill in the art will recognize that modifications and improvements are within the scope and spirit of the invention. The invention having now been described by way of written description, those of skill in the art will recognize that the invention can be practiced in a variety of embodiments and that the foregoing description are for purposes of illustration and not limitation of the following claims.

### REFERENCES

Abrahams et al. (1996) Proc. Natl. Acad. Sci. USA 93(18):9420-9424, “The structure of bovine F₁-ATPase complexed with the peptide antibiotic efrepeption.”
Adams et al. (1998) U.S. Pat. No. 5,780,454
Adams et al. (2000) U.S. Pat. No. 6,083,903
Adams et al. (2001) U.S. Pat. No. 6,297,217 B1
Adams et al. (2003) U.S. Pat. No. 6,617,317 B1
Adams et al. (2004) U.S. Pat. No. 6,747,150 B2
Adams et al. (2005), WO 2005/063714.
Bagatell et al. (2001) Clin. Cancer Res. 7:2076-2084, “Destabilization of Steroid Receptors by Heat Shock Protein 90-binding Drugs: A Ligand-Independent Approach to Hormonal Therapy of Breast Cancer.”
Bagchi et al. (1997) U.S. Pat. No. 5,662,883.
Banerji et al. (2005) J. Clin. Oncol. 23(1):4152-4161, “Phase I Pharmacokinetic and Pharmacodynamic Study of 17-Allylamino, 17-Demethoxygeldanamycin in Patients with Advanced Malignancies.”
Bladé et al. (1998) Br. J. Haematol. 102(5):1115-23, “Criteria for Evaluating Disease Response and Progression in Patients with Multiple Myeloma Treated by High-dose Therapy and Haemopoietic Stem Cell Transplantation.”
Boni et al. (1997) U.S. Pat. No. 5,683,715.
Bosch et al. (1996) U.S. Pat. No. 5,510,118.
Burger et al. (2004) Anti-Cancer Drugs 15 (4):377-387, “17-(Allylamino)-17-demethoxygeldanamycin activity in human melanoma models.”
Chen et al. (2005) Cancer Chemother. Pharmacol. 55:237-243, “Population pharmacokinetic analysis of 17-(allylamino)-17-demethoxygeldanamycin (17AAG) in adult patients with advanced malignancies.”
Cusack, et al. (2005) ASCO 2005 Gastrointestinal Cancer Symp., Abstract No. 276, “NPI-0052, a novel orally administered marine product that promotes chemosensitivity in a colon cancer xenograft model via proteasome inhibition.”
De Castro (1996) U.S. Pat. No. 5,534,270.
Desai et al. (2006) WO 2006/034147 A2.
Egorin et al. (1998) Cancer Res. 58:2385-2396, “Metabolism of 17-(allylamino)-17-demethoxygeldanamycin (NSC 330507) by murine and human hepatic preparations.”
Goetz et al. (2005) J. Clin. Oncol. 23(6):1078-1087, “Phase I trial of 17-allylamino-17-demethoxygeldanamycin in patients with advanced cancer.”
Grem et al. (2005), J. Clin. Oncol. 23(9):1885-93, “Phase I and pharmacologic study of 17-(allylamino)-17-demethoxygeldanamycin in adult patients with solid tumors.”
Gupta (2004) U.S. Pat. No. 6,713,446 B2
Hideshima et al. (2001) Cancer Res. 61, 3071-6, “The Proteasome Inhibitor PS-341 Inhibits Growth, Induces Apoptosis, and Overcomes Drug Resistance in Human Multiple Myelome Cells.”
Hostein et al. (2001) Cancer Res. 61:4003-4009, “Inhibition of Signal Transduction by the Hsp90 Inhibitor 17-Allylamino-17-demethoxygeldanamycin Results in Cytostasis and Apoptosis”
Isaacs et al. (2006) PCT application no. PCT/US2006/007210.
Jiang and Shapiro. (2002) Proc. 93rd Ann Meet Am Assoc Cancer Res. Abstract 1645, “17-AAG induces Rb-dependent G1 arrest in lung cancer cell lines.”
Kadlcikova, et al. (2004) Intl. J. Exp. Pathol. 85(6):365-371, “Effects of proteasome inhibitors MG132, ZL3VS, and AdaAhx3L3VS on protein metabolism in septic rats.”
Kisselev and Goldberg (2001) Chem. Biol. 8:739-758, “Proteasome inhibitors: from research tools to drug candidates.”
Lambert et al. (2000) WO 00/71163.
Lightcap et al. (2000) Clin. Chem. 46 (5), 673-683, “Proteasome Inhibition Measurements: Clinical Application.”
Mansfield et al. (2006) US 2006/0067953 A1.
Mitsiades et al. (2001) Blood 98:795-804, “TRAIL/Apo21 ligand selectively induces apoptosis and overcomes drug resistance in multiple myeloma: therapeutic applications.”
Mitsiades et al. (2003) Cancer Res. 63(20):6689-96, “Fluorescence Imaging of Multiple Myeloma Cells in a Clinically Relevant SCID/NOD in Vivo Model: Biologic and Clinical Implications.”
Mitsiades et al. (2006) Blood 107 (3), 1092-1100, “Antimyeloma activity of heat shock protein-90 inhibition.”
Munster et al. (2001) Proc. Am. Soc. Clin. Oncol. 20:Abstract 327, “Phase I Trial of 17-(allylamino)-17-Demethoxygeldanamycin (17-AAG) in Patients (Pts) with Advanced Solid Malignancies.”
National Cancer Institute (2003) Common Terminology Criteria for Adverse Events v3.0 (CTCAE).
Nguyen et al. (2000) Ann. Thorac. Surg. 70:1853-1860, “Modulation of metastasis phenotypes of non-small cell lung cancer cells by 17-allylamino 17-demethoxy geldanamycin.”
Nimmanapalli et al. (2001) Cancer Res. 61:1799-1804, “Geldanamycin and Its Analogue 17-Allylamino-17-demethoxygeldanamycin Lowers Bcr-Abl Levels and Induces Apoptosis and Differentiation of Bcr-Abl-positive Human Leukemic Blasts.”
Quay et al. (1998), WO 98/30205.
Rahman et al. (1995) U.S. Pat. No. 5,424,073.
Richardson et al. (2003a) N. Eng. J. Med. 348, 2609-17, “A Phase 2 Study of Bortezomib in Relapsed, Refractory Myeloma.”
Richardson et al. (2003b) Cancer Control 10(5):361-369, “Bortezomib (PS-341): a novel, first-in-class proteasome inhibitor for the treatment of multiple myeloma and other cancers.”
Sasaki et al. (1979), J. Antibiotics 32 (8), 849-851, “Growth inhibition of virus transformed cells in vitro and antitumor activity in vivo of geldanamycin and its derivatives.”
Sasaki et al. (1981), U.S. Pat. No. 4,261,989.
Schnur (1995), U.S. Pat. No. 5,387,584.
Schnur et al. (1999), U.S. Pat. No. 5,932,566.
Schulte and Neckers. (1998) Cancer Chemother. Pharmacol. 42:273-279, “The benzoquinone ansamycin 17-allylamino-17-demethoxygeldanamycin binds to HSP90 and shares important biologic activities with geldanamycin.”
Shah et al. (2002) Br. J. Clin. Pharmacol. 54(3):269-276, “Early clinical experience with the novel proteasome inhibitor PS-519.”
Sonntag et al. (2001) WO 01/10412 A1.
Straubinger et al. (1995) U.S. Pat. No. 5,415,869.
Tabibi et al. (2004) U.S. Pat. No. 6,682,758 B1
Ulm et al. (2003) WO 03/086381 A1.
Ulm et al. (2004) WO 2004/082676 A1.
Wermuth (2003), “Designing Prodrugs and Bioprecursors,” in Wermuth, ed., The Practice of Medicinal Chemistry, 2nd Ed., pp. 561-586 (Academic Press 2003).
Zhong et al. (2005) US 2005/0256097 A1

What is claimed is:

1. A method of treating multiple myeloma (MM) in a subject in need of such treatment, comprising the step of administering to said subject a therapeutically effective dose of 17-allylamino-17-demethoxy-geldanamycin (17-AAG) or 17-aminogeldanamycin (17-AG) or a prodrug of either 17-AAG or 17-AG, and a therapeutically effective dose of a proteasome inhibitor, and optionally repeating said step until no further therapeutic benefit is obtained.

2. A method of treating MM in a subject in need of such treatment, comprising the step of administering multiple doses of 17-AAG or 17-AG or a prodrug of either to said subject over a time period of at least 2 weeks, wherein each such dose is in the range of about 100 to about 340 mg/m² of 17-AAG, or an equivalent amount of 17-AG or a 17-AAG prodrug or 17-AG prodrug, and multiple doses of a proteasome inhibitor, wherein said proteasome inhibitor is bortezomib and each such dose is at least about 1 mg/m².

3. The method of claim 2, wherein each such dose of 17-AAG is in the range of about 150 to about 340 mg/m or an equivalent amount of 17-AG or a prodrug of 17-AAG or 17-AG.

4. The method of claim 2, wherein said dose of is administered twice weekly for at least two weeks.

5. The method of claim 4, wherein said dose is administered twice weekly for at least two weeks in a three week period.

6. The method of claim 5, wherein multiple cycles of treatment are administered to the subject, wherein each cycle of treatment comprises of said dose administered twice weekly for at least two weeks in a three week period.

7. A method of treating MM in a subject in need of such treatment, comprising the step of administering a therapeutically effective dose of a proteasome inhibitor and a therapeutically effective dose of 17-AAG or a prodrug of 17-AAG that results in an AUCₜₒₜₐₗ of 17-AAG per dose in the range of about 2,300 to about 19,000 ng/mL*h.

8. The method of claim 7, wherein said dose of 17-AAG is administered at a rate and frequency such that the Cₘₐₓ of 17-AAG does not exceed 9,600 ng/mL.

9. The method of claim 7, wherein said dose of 17-AAG is administered at a rate and frequency such that the Cₘₐₓ of 17-AAG is greater than 1,300 ng/mL.

10. The method of claim 9, wherein said dose of 17-AAG is administered at a rate and frequency such that the Cₘₐₓ of 17-AAG is greater than 1,800 ng/mL.

11. The method of claim 7, wherein said dose of 17-AAG is administered at a rate and frequency such that the Cₘₐₓ of 17-AAG is greater than 1,300 but does not exceed 9,600 ng/mL.

12. The method of claim 7, wherein said dose of 17-AAG is administered at a rate and frequency such that the Cₘₐₓ of 17-AAG is greater than 1,800 but does not exceed 9,600 ng/mL.

13. A method of treating MM in a subject in need of such treatment, comprising the step of administering to said subject a therapeutically effective dose of a proteasome inhibitor and a therapeutically effective dose of 17-AG or a prodrug of 17-AG that results in an AUCₜₒₜₐₗ of 17-AG per dose in the range of about 800 to about 17,000 ng/mL*h.

14. The method of claim 13, wherein said dose of 17-AG is administered at a rate and frequency such that the Cₘₐₓ of 17-AAG does not exceed 1,400 ng/mL.

15. The method of claim 13, wherein said dose of 17-AG is administered at a rate and frequency such that the Cₘₐₓ of 17-AAG is greater than 140 ng/mL.

16. The method of claim 15, wherein said dose of 17-AG is administered at a rate and frequency such that the Cₘₐₓ of 17-AAG is greater than 230 ng/mL.

17. The method of claim 13, wherein said dose of 17-AG is administered at a rate and frequency such that the Cₘₐₓ of 17-AAG is greater than 140 but does not exceed 1,400 ng/mL.

18. The method of claim 17, wherein said dose of 17-AG is administered at a rate and frequency such that the Cₘₐₓ of 17-AAG is greater than 230 but does not exceed 1,400 ng/mL.

19. A method of treating MM in a subject in need of such treatment, comprising the step of administering to said subject a therapeutically effective dose of a proteasome inhibitor and a therapeutically effective dose of 17-AAG, a prodrug of 17-AAG, 17-AG, or a prodrug of 17-AG that results in a combined AUCₜₒₜₐₗ of 17-AAG and 17-AG per dose in the range of about 3,500 to about 35,000 ng/mL*h.

20. The method of claim 19, wherein said dose of 17-AAG, a prodrug of 17-AAG, 17-AG, or a prodrug of 17-AG is administered at a rate and frequency such that the Cₘₐₓ of 17-AAG does not exceed 9,600 ng/mL or the Cₘₐₓ of 17-AG does not exceed 1,400 ng/mL.

21. The method of claim 19, wherein said dose of 17-AAG, a prodrug of 17-AAG, 17-AG, or a prodrug of 17-AG is administered at a rate and frequency such that the Cₘₐₓ of 17-AAG is greater than 1,300 ng/mL or the Cₘₐₓ of 17-AG is greater than 140 ng/mL.

22. The method of claim 21, wherein said dose is administered at a rate and frequency such that the Cₘₐₓ of 17-AAG is greater than 1,800 ng/mL or the Cₘₐₓ of 17-AG is greater than 230 ng/mL.

23. The method of claim 19, wherein said dose of 17-AAG, a prodrug of 17-AAG, 17-AG, or a prodrug of 17-AG is administered at a rate and frequency such that the Cₘₐₓ of 17-AAG is greater than 1,300 but does not exceed 9,600 ng/mL or the Cₘₐₓ of 17-AG is greater than 140 but does not exceed 1,400 ng/mL.

24. The method of claim 23, wherein said dose is administered at a rate and frequency such that the Cₘₐₓ of 17-AAG is greater than 1,800 but does not exceed 9,600 ng/mL or the Cₘₐₓ of 17-AG is greater than 230 ng/mL but does not exceed 1,400 ng/mL.

25. A method of treating MM in a subject in need of such treatment, comprising the step of administering to said subject a therapeutically effective dose of a proteasome inhibitor and a therapeutically effective dose of 17-AAG or a prodrug of 17-AAG that results in a Terminal T_{1/2} of 17-AAG in the range of 1.6 h to 5.6 h.

26. The method of claim 25, wherein said dose of 17-AAG or a prodrug of 17-AAG results in an AUCₜₒₜₐₗ of 17-AAG per dose in the range of about 2,300 to about 19,000 ng/mL*h.

27. A method of treating MM in a subject in need of such treatment, comprising the step of administering to said subject a therapeutically effective dose of a proteasome inhibitor and a therapeutically effective dose of 17-AG or a prodrug of 17-AG that results in a Terminal T_{1/2} of 17-AG in the range of 3.7 h to 9.1 h.

28. The method of claim 27, wherein said dose of 17-AG or a prodrug of 17-AG results in an AUCₜₒₜₐₗ of 17-AG per dose in the range of about 800 to about 17,000 ng/mL*h.

29. A method of treating MM in a subject in need of such treatment, comprising the step of administering to said subject a therapeutically effective dose of a proteasome inhibitor and a therapeutically effective dose of 17-AAG or a prodrug of 17-AAG that results in a Volume of distribution V_{Z} of 17-AAG in the range of 56 L to 250 L.

30. The method of claim 29, wherein said dose of 17-AAG or a prodrug of 17-AAG results in an AUCₜₒₜₐₗ of 17-AG per dose in the range of about 2,300 to about 19,000 ng/mL*h.

31. A method of treating MM in a subject in need of such treatment, comprising the step of administering to said subject a therapeutically effective dose of a proteasome inhibitor and a therapeutically effective dose of 17-AAG or a prodrug of 17-AAG that results in a Clearance of 17-AAG in the range of 13 to 85 L/h.

32. The method of claim 31, wherein said dose of 17-AAG or a prodrug of 17-AAG results in an AUCₜₒₜₐₗ of 17-AG per dose in the range of about 2,300 to about 19,000 ng/mL*h.

33. A method of treating MM in a subject in need of such treatment, comprising the step of administering to said subject a therapeutically effective dose of a proteasome inhibitor and a therapeutically effective dose of 17-AAG or a prodrug of 17-AAG that results in a Volume of distribution V_{SS} of 17-AAG in the range of 96 to 250 L.

34. The method of claim 33, wherein said dose of 17-AAG or a prodrug of 17-AAG results in an AUCₜₒₜₐₗ of 17-AG per dose in the range of about 2,300 to about 19,000 ng/mL*h.

35. The method of claim 2, wherein said each dose of bortezomib is of the range of about 1.0 to about 1.3 mg/m².

36. The method of claim 1, wherein said proteasome inhibitor is a peptide aldehyde.

37. The method of claim 1, wherein said peptide aldehyde is a peptide boronate.

38. The method of claim 37, wherein said peptide boronate is a dipeptide boronic acid.

39. The method of claim 38, wherein said dipeptide boronic acid is bortezomib.

40. The method of claim 1, wherein said administering step results in an induction of HSP70 in peripheral blood mononuclear cells of said subject.

41. The method of claim 40, wherein said induction of HSP70 is observable one day after said administering step.

42. The method of claim 1, wherein said administering step results in an increase of apoptosis of CD138⁺ cells among the bone marrow aspirate cells of said subject.

43. The method of claim 42, wherein said increase of apoptosis of CD138⁺ cells is observable four hours after said administering step.

44. The method of claim 1, wherein said administering step results in a decrease of total AKT in bone marrow aspirate cells of said subject.

45. The method of claim 44, wherein said decrease of total AKT is observable four hours after said admnistering step.
